(19) United States
(12) Patent Application Publication
(10) Pub. No.: US 2023/0200689 A1
(43) Pub. Date: Jun. 29, 2023

(54) Systems and Methods for Detecting the Presence of an Analyte in a Sample

(71) Applicant: ADL Diagnostics, Inc., Woodinville, WA (US)

(72) Inventors: Robert G. Atkinson, Woodinville, WA (US); Minh Duong, Woodinville, WA (US)

(21) Appl. No.: 17/980,307

(22) Filed: Nov. 3, 2022

Related U.S. Application Data
(60) Provisional application No. 63/275,758, filed on Nov. 4, 2021.

(51) Int. Cl. A61B 5/145; A61B 5/00

(52) U.S. Cl. CPC A61B 5/14546; A61B 5/0017

(57) ABSTRACT

The device includes a well configured to receive a reaction tube containing an analyte, and a receiver configured to receive a near-field communication signal from a near-field communication chip coupled to the reaction tube, the near-field communication signal including information for determining a parameter associated with a type of test to be performed on the analyte by the device. The device further includes a light emitting source configured to emit an excitation light at a wavelength to illuminate the analyte in the reaction tube, an optical detector configured to receive an emission light in response to the analyte being illuminated by the excitation light, and a processor operably coupled to the receiver and the light emitting source, the processor configured to select the wavelength of the emission light based on the parameter.

### CROSS REFERENCE TO RELATED APPLICATION

The application claims priority to and the benefit of U.S. Provisional Patent Application No. 63/275,758, filed on Nov. 4, 2021, the entire disclosure of which is hereby incorporated by reference.

### TECHNICAL FIELD

The present disclosure relates generally to bioanalytical systems suitable for detecting one or more analytes. Embodiments described herein are particularly well suited for instruments capable of performing a variety of assays, such as selectively amplifying a variety of a target analytes and determining whether a particular target analyte is present in a sample. Some embodiments relate to reaction tubes encoded to identify the appropriate assay such that the instrument automatically runs the correct assay for the sample contained therein.

### BACKGROUND

A number of diagnostic and analytic techniques have been developed to detect the presence of proteins, DNA, or other suitable biomarkers, for example, those associated with severe acute respiratory syndrome coronavirus 2 (SARS-CoV-2), influenza, human immunodeficiency virus (HIV), Mycobacterium tuberculosis, etc. Many such techniques are designed to amplify a target analyte for a predetermined period of time and then determine whether a quantity of the target analyte is detectable and/or exceeds a predetermined threshold that indicates a “positive” result. Such techniques are time consuming, as they must generally be run to completion, or at least until a signal associated with the target analyte crosses a pre-defined threshold. The lengthy run time for such techniques has contributed to significant delays in obtaining test results. For example, in many cases, the wait time to obtain a COVID-19 test result is 7-10 days.

Further, known instruments capable of performing different types of assays typically require highly skilled operators. For example, the operators are required to instruct a device on detailed technical parameters to amplify a target analyte. In some instances, the operator may further need to interpret the results. A need exists for systems and methods that are capable of performing multiple assays and that automatically select parameters for an assay based on a type of assay that needs to be performed.

Systems and methods described herein are well suited for performing automatic selection of assay parameters based on the type of the assay that needs to be performed. Further, by automating the analysis of an analyte, the system and methods described herein facilitate “rapid” testing of large number of samples, which can significantly contribute to curbing the spread infections.

### SUMMARY

Consistent with a disclosed embodiment, a device is provided. The device includes a well configured to receive a reaction tube containing an analyte and a receiver configured to receive a near-field communication signal from a near-field communication chip coupled to the reaction tube, the near-field communication signal including information for determining a parameter associated with a type of test to be performed on the analyte by the device. The device further includes a light emitting source configured to emit an excitation light at a wavelength to illuminate the analyte in the reaction tube, an optical detector configured to receive an emission light in response to the analyte being illuminated by the excitation light, and a processor operably coupled to the receiver and the light emitting source, the processor configured to select the wavelength of the emission light based on the parameter.

Consistent with another disclosed embodiment, a device includes a heat block defining a well configured to receive a reaction tube containing an analyte and a receiver configured to receive a near-field communication signal from a near-field communication chip coupled to the reaction tube, the near-field communication signal including information for determining a parameter associated with a type of test to be performed on the analyte by the device. Further, the device includes a processor operably coupled to the heat block and the receiver. The processor is configured to control a temperature of the heat block based on the parameter such that the analyte is amplified and determine at least one of a quantity or a concentration of the analyte after amplifying the analyte.

Consistent with another disclosed embodiment, a reaction tube is provided. The reaction tube is configured to be inserted in an enclosure of a device for performing a test to determine at least one of a quantity or a concentration of an analyte. The reaction tube includes a closed bottom portion that is at least partially transparent to excitation light at an excitation wavelength and to emission light at an emitted wavelength and T top tube member opposite the bottom portion includes a near-field communication chip configured to communicate a near-field communication signal to a receiver of a device configured to perform one or more assays. The near-field communication chip can store parameters such that the signal can include instructions to the device as to how to conduct an assay of the analyte.

Consistent with another disclosed embodiment, a system is provided. The system includes a device. The device includes a heat block defining a well, the heat block configured control a temperature of at least one reaction tube from a plurality of reaction tubes, and a receiver configured to receive any near-field communication signals from a reaction tube disposed in the well. The near-field communication signal can include one or more parameters for performing an assay on a sample contained in the reaction tube. In this way, different reaction tubes, having different chips and configured to contain different samples/be subject to different assays can send different parameters to the device. Further, the device includes a light emitting source configured to emit an excitation light to illuminate the sample in the reaction tube, an optical detector configured to receive an emission signal in response to the sample being illuminated by the excitation light, and a processor operably coupled to the receiver and configured to control the heating block, the light emitting source, and the optical detector based on parameters received from the reaction tube.

### BRIEF DESCRIPTION OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIG. 1 is an example system for selectively amplifying a target analyte and determining whether the target analyte is present or absent in a sample, according to an embodiment.

FIGS. 2A-2F are example views of a reaction tube disposed in a well, according to an embodiment.

FIGS. 3A-3D are other example views of a reaction tube, according to an embodiment.

FIG. 4 is another example system for selectively amplifying a target analyte and determining whether the target analyte is present or absent in a sample, according to an embodiment.

FIG. 5 is an example device and a reaction tube, according to an embodiment.

FIGS. 6A and 6B are example views of a well of an example device, according to an embodiment.

FIGS. 7A-7C are example views of a device, according to an embodiment.

FIGS. 8A-1 and 8A-2 are examples views of a reaction tube, according to an embodiment.

FIGS. 8B-8F are example views of an alternative reaction tube, according to an embodiment.

FIG. 9 is an example cross-sectional view of a device, according to an embodiment.

FIG. 10 is a flow chart of a method of detecting an analyte, according to an embodiment.

FIG. 11 is an experimental data from an analysis of an example sample, according to an embodiment.

FIGS. 12A-12H are example steps of a process of analyzing an analyte using a test kit, according to an embodiment.

FIGS. 13A-13H are example illustrations of an alternative reaction tube, according to an embodiment.

### DEFINITIONS

Unless otherwise defined herein, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described herein, are those well-known and commonly used in the art.

As used herein, the following terms have the meanings ascribed to them unless specified otherwise.

The term “including” is used to mean “including but not limited to.” “Including” and “including but not limited to” are used interchangeably.

The words “a” and “an” denote one or more, unless specifically noted.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length. In any embodiment discussed in the context of a numerical value used in conjunction with the term “about,” it is specifically contemplated that the term about can be omitted.

Unless the context requires otherwise, throughout the present specification and claims, the word “comprise” and variations thereof, such as, “comprises” and “comprising” are to be construed in an open, inclusive sense, that is as “including, but not limited to.”

By “consisting of” is meant including, and limited to, whatever follows the phrase “consisting of” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used herein, the term “sample” refers to a composition that contains an analyte or analytes. A sample can be heterogeneous, containing a variety of components or homogenous, containing one component. In some instances, a sample can be naturally occurring, a biological material, and/or a man-made material. Furthermore, a sample can be in a native or denatured form. In certain embodiments, the sample is a biological sample. In some instances, a sample can be a single cell (or contents of a single cell) or multiple cells (or contents of multiple cells), a saliva sample, a mucous sample, a blood sample, a tissue sample, a skin sample, a urine sample, a water sample, and/or a soil sample. In some instances, a sample can be from a living organism, such as a eukaryote, prokaryote, mammal, human, yeast, and/or bacterium or the sample can be from a virus. In some embodiments, a sample can be a food product or a beverage product. In some embodiments, a sample can be a swab of a surface, e.g., a swab of a food preparation surface or a container.

As used herein, the term “analyte” refers to any molecule or compound to be detected as described herein. Suitable analytes can include but are not limited to, small chemical molecules and/or biomolecules, such as, for example, environmental molecules, clinical molecules, chemicals, and pollutants. More specifically, such chemical molecules and/or biomolecules can include but are not limited to pesticides, insecticides, toxins, therapeutic and/or abused drugs, hormones, antibiotics, antibodies, organic materials, proteins (e.g., enzymes, immunoglobulins, and/or glycoproteins), nucleic acids (e.g., DNA and/or RNA), lipids, lectins, carbohydrates, whole cells (e.g., prokaryotic cells such as pathogenic bacteria and/or eukaryotic cells such as mammalian tumor cells), viruses, spores, polysaccharides, glycoproteins, metabolites, cofactors, nucleotides, polynucleotides, transition state analogs, inhibitors, nutrients, electrolytes, growth factors and other biomolecules and/or non-biomolecules, as well as fragments and combinations thereof. Some analytes described herein can be proteins such as enzymes, drugs, cells, antibodies, antigens, cellular membrane antigens, and/or receptors or their ligands (e.g., neural receptors or their ligands, hormonal receptors or their ligands, nutrient receptors or their ligands, and/or cell surface receptors or their ligands). In particular embodiments, an analyte is or is associated with an infectious or pathological agent, such as, e.g., a bacterium, virus, yeast, or fungus.

As used herein, the term “protein” refers to proteins, polypeptides, oligopeptides, peptides, and analogs, including proteins containing non-naturally occurring amino acids and amino acid analogs, and peptidomimetic structures. The term “protein” also refers to proteins, polypeptides, oligopeptides, peptides, and analogs.

### DETAILED DESCRIPTION

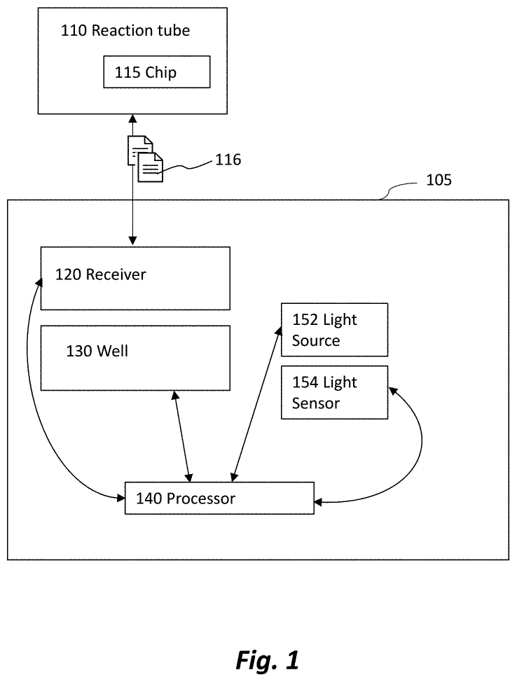

The present disclosure describes systems, apparatuses, and methods for selectively amplifying a target analyte and determining whether the target analyte is present or absent in a sample based on a change of a signal associated with the quantity of the analyte. Consistent with disclosed embodiments, a device for determining the target analyte is provided. An example device 105 is illustrated in FIG. 1. Device 105 includes a well 130 configured to receive a reaction tube 110 containing a sample with an analyte. Well 130 may be any suitable opening for receiving at least a portion of reaction tube 110. In an example embodiment, well 130 may have a shape that substantially similar to the shape of at least a portion of reaction tube 110. In an example embodiment, well 130 includes at least a tube holding member and an opening in which reaction tube 110 may be inserted. In an example embodiment, well 130 includes an enclosure having walls, with the walls of enclosure configured to be adjacent (at least partially) to walls of reaction tube 110. Exemplary methods for selectively amplifying a target analyte and determining whether the target analyte is present or absent in a sample based on a change of a signal associated with the quantity of the analyte are described in further detail in U.S. patent application Ser. No. 17/666,338, filed on Feb. 7, 2022, and PCT Application No. PCT/US22/15512 designating the United States, filed on Feb. 7, 2022, which are incorporated by reference herein in their entireties.

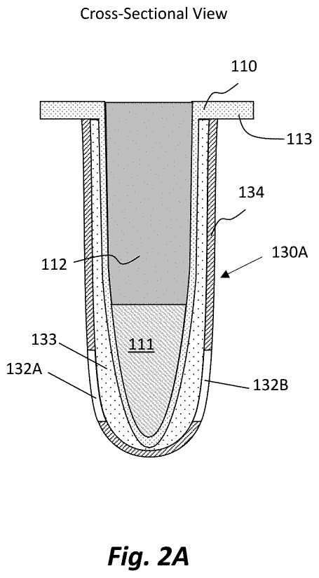

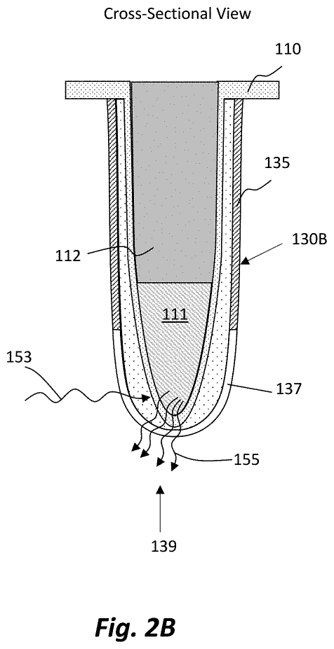

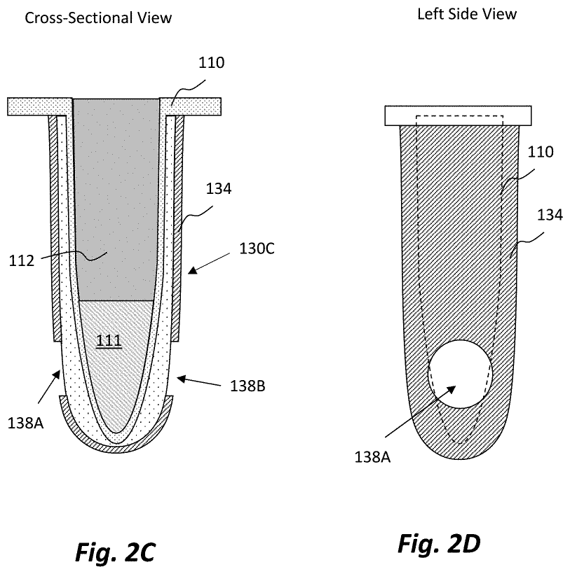

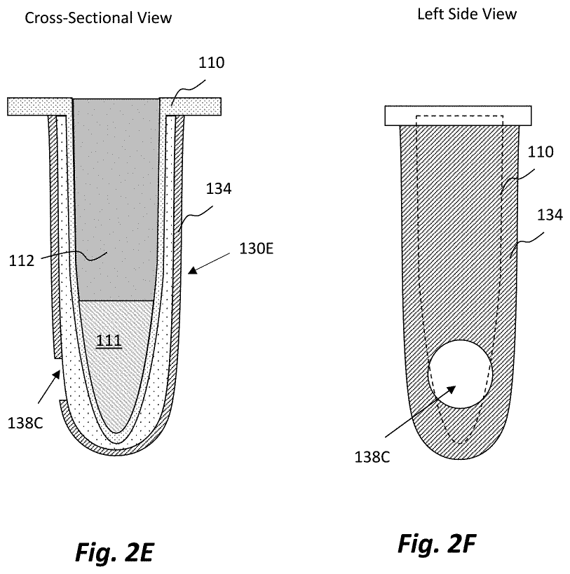

Example embodiments of a well configured to receive (and shown as containing) a reaction tube 110 are shown in FIGS. 2A-2D. FIGS. 2A-2C, and 2E show cross-sectional views of example reaction tube disposed in a well while FIGS. 2D and 2F shows a side view of an example reaction tube disposed in a well. Consistent with one embodiment, FIG. 2A shows a well 130A having walls 134 forming an enclosure 133. Enclosure 133 is configured to receive reaction tube 110, as shown in FIG. 2A. In an example embodiment, reaction tube 110 forms an enclosure 112 containing a sample 111 with an analyte, which, for example, may include a liquid. In an example embodiment, reaction tube 110 may include a rim 113 that is configured to rest over a top portion of well 130A. In an example embodiment, as shown in FIG. 2A, well 130A may include a first window 132A and a second window 132B. In an example embodiment, at least one of first window 132A or second window 132B are configured to be transparent to a light emitted by a light emitting source. In an example embodiment, the light emitting source is configured to emit light at a target excitation wavelength used to illuminate sample 111. In an example embodiment, when the light emitting source is adjacent to windows 132A (or window 132B), the window 132A (or window 132B) is configured to be transparent to the target excitation wavelength. In various embodiments, at least one window (window 132A or 132B) is configured to be transparent to a wavelength of light emitted by sample 111 in response to sample 111 being illuminated by the excitation light. In an example embodiment, if an optical detector is present adjacent to window 132A (or window 132B), window 132A (or window 132B) is configured to be transparent to the wavelength of light emitted by sample 111. In some cases, windows 132A and/or 132B may be transparent to both the target excitation wavelength and the wavelength of light emitted by sample 111. In an example embodiment, walls 134 may be made from any suitable material (e.g., plastic, metal, glass, ceramics, etc.) capable of supporting reaction tube 110, while windows 132A and 132B may be made of glass, quartz, or any other suitable light transparent material (e.g., sapphire, MgF₂, transparent plastic, and the like). In some cases, window 132B may be configured to partially absorb a target excitation wavelength of light while transmitting (at least partially) light emitted by sample 111 (e.g., window 132B may provide for light filtering to further separate target excitation wavelength of light from the light emitted by sample 111).

FIG. 2B shows another embodiment of well 130B, including window 137 extending through the bottom part of well 130B. In an example embodiment, window 137 may be made from any suitable light transparent material similar to material used for windows 132A and 132B. In an example embodiment, window 137 may be transparent to target excitation wavelength 153 emitted by a light emitting device and to a wavelength of light 155 emitted by sample 111. In an example embodiment, light 155 may be measured at location 139, as shown in FIG. 2B.

FIG. 2C shows another embodiment of well 130C wherein instead of windows 132A and 132B, well 130C contains openings 138A and 138B. FIG. 2D shows a side view of opening of 138A formed in wall 134, which, in an example embodiment, may be circular (or may be of any suitable shape). Reaction tube 110 is shown in FIG. 2D using a dashed line. FIG. 2E shows an example embodiment of a well 130E with only one opening 138C. Alternatively, to opening 138C, well 130E may include a light transparent window similar to windows 132A or 132B. FIG. 2F shows a side view (left side) of well 130E, which may be similar to side view shown in FIG. 2D (e.g., side view shown in FIG. 2F includes opening 138C and shows walls 134 and reaction tube 110). In an example embodiment single opening 138C may be used for receiving a target excitation wavelength emitted by a light emitting device adjacent to opening 138C and for transmitting light emitted by sample 111 to an optical detector that may also be adjacent to opening 138C, and also adjacent to the light emitting device.

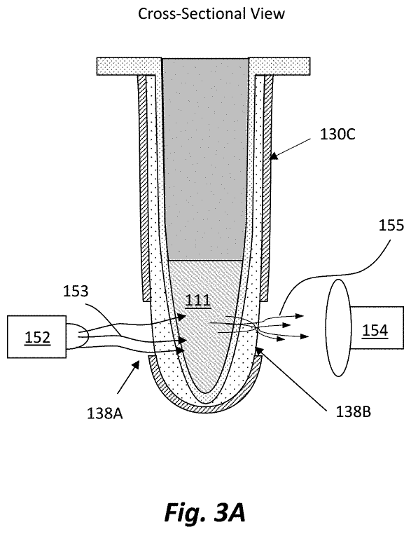

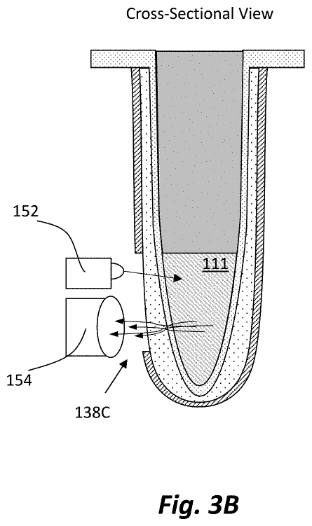

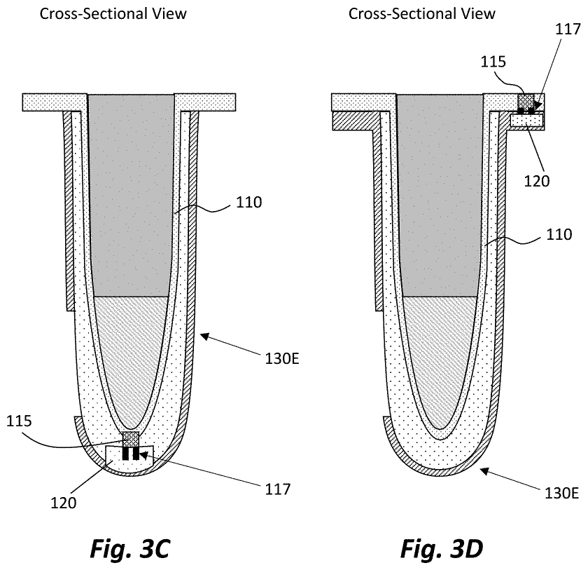

FIGS. 3A and 3B show example embodiments of device 105 that includes light emitting source 152 and an optical detector 154. For well 130C, as shown in FIG. 3A, light emitting source 152 may be placed next to opening 138A (or next to window 132A for well 130B), while optical detector 154 may be placed next to opening 138B (or next to window 132B for well 130B). In an example embodiment, light emitting source 152 is configured to emit a target excitation wavelength 153 of light and optical detector 154 is configured to detect a wavelength of light 155 emitted from sample 111. As shown in FIG. 3A, emitted light 155 is observed through opening 138B, while target excitation wavelength 153 is transmitted to analyte via opening 138A.

In an example embodiment, light emitting source 152 may be any suitable source such as light emitting diode, laser diode, solid state laser, chemical laser, incandescent light, fluorescent light, xenon light, or any other suitable light emitting source. In an example embodiment, light emitting source 152 may emit light at a particular peak wavelength. In some cases, a full width at half maximum (FWHM) for light emitted spectra of light emitting source 152 may be a few tens of nanometers for peak wavelengths in a visible spectrum. For example, for peak wavelength of about 400-700 nm, FWHM may be in a range of 1-50 nm. Alternatively, light emitting source 152 may emit at a plurality of peak wavelengths and may be configured to emit at a selected one or more peak wavelengths based on received input commands. Additionally, the FWHM of a peak wavelength may be configured to be narrowed or expanded based on the received input commands. Alternatively, light emitting source 152 may be a broad-spectrum source. Further, light emitting source 152 may emit light continuously for a given duration of time, may emit a pulse of light, or a periodic train of pulses.

In some cases, light emitting source 152 may include adjustable (e.g., movable) optical element (or a plurality of optical elements) configured to adjust parameters of light 153 (e.g., focus light 153 over a region of sample 111). For example, the optical element of light emitting source 152 may be a shutter, a movable lens for focusing 153, a prism for further filtering a wavelength of light 153, a diffraction grating, a system of movable lenses, a movable mirror, and the like. In an example embodiment, the optical element may be moved or rotated via a suitable mechanism (e.g., an electrical motor). In other embodiments, the light emitting source 152 may be located away from the opening 138A, and light from the light emitting source 152 can be transmitted to the opening 138A (and the region of sample 111) via a fiber optic or other optical pathway. Such an embodiment may be particularly well suited to multi-well devices (not shown), as a single light emitting source can transmit light to multiple wells via fiber optic paths.

In various embodiments, optical detector 154 may be any suitable optical detector such as photodiode, avalanche diode, CMOS sensor, or any other suitable sensor (e.g., one or more field effect transistors) for detecting emitted light 155. In some cases, optical detector 154 may include a plurality of optical sensors, each one from the plurality of optical sensors configured to sense light in a corresponding wavelength range. For example, optical detector 154 may include a first sensor configured to sense light in a first wavelength range and a second sensor configured to sense light in a second wavelength range. In some cases, the first and the second wavelength ranges may overlap or partially overlap. In other cases, the first and second wavelength ranges may not overlap. In some embodiments, the optical detector 154 may be located away from opening 138B, and the light emanating from the region of the sample can be transmitted to the optical detector 154 via a fiber optic or other optical pathway.

Returning to FIG. 1, in an example embodiment, device 105 may also include a processor 140 operably coupled to receiver 120, light emitting source 152 and optical detector 154. In various embodiments, processor 140 is configured to exchange data with receiver 120, source 152 and detector 154. For instance, receiver 120 may send data 116, and data 116 may be used by processor 140 for determining operational parameters for source 152 and detector 154. Among possible operational parameters, processor 140 may determine a wavelength (or a plurality of wavelengths) at which light emitting source 152 is configured to emit light 153, intensity of light 153, duration of time during which a pulse of light 153 is emitted, a number of pulses of light 153 emitted, FWHM values for light 153, or any other characteristics associated with emitted light 153. Additionally, processor 140 is configured to activate detector 154 or select one of the plurality of sensors forming detector 154 for sensing light 155 emitted by sample 111 when sample 111 is illuminated by light 153.

Further, processor 140 may determine when to emit excitation wavelength 153 and when to measure the emitted light 155. For example, to improve measurements associated with emitted light 155, optical detector may be activated at a time T₁ which is slightly later than time T₀ at which light emitting source emits excitation wavelength 153. In an example embodiment, ΔT=(T₁−T₀) may be in a range of a few microseconds to a few milliseconds, depending on emission properties of analyte. In some cases, T₁=T₀, and in other cases, T₀>T₁ (i.e., optical detector may be activated prior to emission of excitation wavelength 153.

In various embodiments, reaction tube 110 may include a chip 115 configured to send and receive electrical signals to a receiver 120 of device 105. In one example embodiment, chip 115 may include data 116 for device 105. Data 116 may describe sequence of steps for testing sample 111. In some cases, data 116 may be used to retrieve a set of instructions for testing sample 111. For example, data 116 may be used to retrieve the set of instructions from a local memory associated with device 105, or data 116 may be used to retrieve the set of instruction from a server connected remotely to device 105. Additionally, or alternatively, data 116 may include at least some of the instructions for testing sample 111.

In an example embodiment, chip 115 may be an active radio frequency identification device (RFID) or a passive RFID (active RFIDs include a power supply, while passive RFIDs draw external power in a form of an electromagnetic signal to transmit data). In instances in which chip 114 is a passive RFID, receiver 120 is configured to supply power to facilitate chip 115 transmitting data to receiver 120 (in some cases, power to chip 115 may be supplied by another device associate with device 105, or another component of device 105 configured to supply power to chip 115). In some cases, when chip 115 is powered externally via suitable electrical connections, reaction tube 110 may be electrically connected to device 105 (e.g., electrical connection between reaction tube 110 and electrical device 105 may be established by inserting tube 110 into device 105). An example embodiment of such communication is shown in FIG. 3C, in which reaction tube 110 includes chip 115 on a bottom portion of tube 115, and chip is electrically connected via connection 117 to receiver 120 for exchanging data (and/or power) with receiver 120. It should be appreciated that chip 115 may be located at any other portion of reaction tube 110. For example, FIG. 3D shows chip 115 located in a rim of reaction tube 110 and connected via connection 117 to receiver 120 located in a top portion of well 130E.

In an example embodiment, chip 115 may be a flexible element in a form of a sticker and is configured to be attached to reaction tube 110. In an example embodiment, for cases when chip 115 is configured to communicate wirelessly with receiver 120 of device 105, chip 115 may be attached to various portions of reaction tube 110 (e.g., chip 115 may be attached to a rim of tube 110, walls of tube 110, and the like). In an example embodiment, chip 115 may be configured to communicate with receiver 120 via a near field communication signal. For example, signal from chip 115 may not be detectable at a distance substantially further than a distance between receiver 120 and chip 115. For example, chip 115 may not be readable further than an inch away from reaction tube 110, further than a few inches away from reaction tube 110, further than a foot away from reaction tube 110, or further than a few feet away from reaction tube 110. In an example embodiment, near-field communication between reaction tube 110 and device 105 may ensure that information stored in chip 115 is not accessible to other devices in vicinity of reaction tube 110 and/or that chips on tubes outside of the device 105 do not transmit information to device 105. In an example embodiment, only device 105 may be configured to obtain data from chip 115. For instance, communication between device 105 and chip 115 may be suitably encrypted (e.g., encrypted using secure shell protocol) to prevent external devices from accessing data 116 residing on chip 115.

In an example embodiment, on a front flat side of a shroud of reaction tube 110 (herein the shroud of reaction tube 110 may be a top portion of reaction tube 110), an NXT NTAG213 integrated circuit (IC) together with a small antenna may be placed to form the NFC tag. The tag may only have a small antenna as it is configured to be read from a short range of about a few centimeters or less (e.g., less than one centimeter). In an example embodiment, a tag may contain tens or hundreds of byte of persistent arbitrary user data. In some cases, tag may store thousands of bytes or more of the data. The data may include the reaction tube identifier, test identification, test parameterization, and/or a cryptographic hash-based message authentication (HMAC). In some cases, it may be desirable that device 105 is configured to refuse to execute assays if a genuine reaction tube containing an authentic tag (e.g., assays that are third-party/knock-off/or pirated), as inauthentic reaction tubes may include incorrect and/or inferior primers or other reagents, which may not produce accurate results, and may reduce confidence in the instrument and/or test results. In some instances, the reaction tube 110 and/or tag may be operable to assure that the tube has been properly stored and/or has not expired. For example, the tag can store a manufactured date and/or an expiration date, and device 105 may be configured to refuse to execute assays if the tube has expired and/or is too old and may return a suitable error message. In addition, or alternatively, the tag can contain or be coupled to an analog or digital sensor capable of determining if a safe storage temperature (high and/or low) of the reaction tube 110 (and any reagents it contains) has been exceeded. Other suitable environmental, storage, and or handling sensors are also possible, such as, for example, drop sensors/accelerometers such that the device 105 can be configured to return a suitable error message if the reaction tube 110 has been dropped, sensors and/or clocks configured to detect when a seal has been broken such that the device 105 can be configured to return a suitable error message if the reaction tube 110 has not been used within a predetermined amount of time after being opened, etc.

In an example embodiment, the tag data is read by having the tag brought in proximity to an NFC receiver. An example receiver (e.g., receiver 120, as shown in FIG. 1) may reside either in the front lip of the lid or under the top of the main chassis, near the lid button mechanism for device 105. Receiver 120 may consist of an antenna and supporting circuitry. An alternative current (AC) coupling from receiver 120's antenna to the tag's antenna provides power to the tag, and the NFC standards define how that AC is modulated to allow the receiver 120's IC and the tag's IC to communicate and, in particular, read the data in the tag.

In certain implementations, RFID and/or NFC chips are advantageous because of their small size and relatively large storage capacity (e.g., dozens or hundreds of bits). It should be appreciated that chip 115 is not limited to passive or active RFIDs. Chip 115 may be any suitable device or an article of manufacture capable of transmitting data to receiver 120 via any suitable means (herein, transmitting data includes at least one of sending data, or receiving data), such as any wireless communication (e.g., near field communication, WiFi communication, any suitable Bluetooth communication, RFID based communication), wired communication, optical wireless (or wired) communication (e.g., via a light signal sending a Morse code), via reading a 2D barcode read by a camera, QR codes, and the like. For instance, chip 115 may include a Datamatrix printed on a sticker that may be attached to reaction tube 110. The sticker may be read by a camera of a device 105.

In some cases, device 105 may also include a light (e.g., an LED light) for illuminating the 2D barcode. In various embodiments, NFC tags are more versatile than QR codes and/or other 2D or 3D barcodes. For example, NFC tags may be reprogrammed, while QR codes remain the same once generated. NFC offers faster, easier, and more secure transactions. Major advantages of NFCs are their storage density and flexibility. Storing different types of information and changing it on a whim is possible without creating a new NFC tag. The owner can simply overwrite the information currently on the NFC tag. Thus, reaction tube 110 containing an NFC chip may be readily reprogrammed for a different assay test.

In some cases, device 105 may also include a light (e.g., an LED light) for illuminating the 2D barcode. In various embodiments, NFC tags are more versatile than QR codes and/or other 2D or 3D barcodes. For example, NFC tags may be reprogrammed, while QR codes remain the same once generated. NFC offers faster, easier, and more secure transactions. Major advantages of NFCs are their storage density and flexibility. Storing different types of information and changing it on a whim is possible without creating a new NFC tag. The owner can simply overwrite the information currently on the NFC tag. Thus, reaction tube 110 containing an NFC chip may be readily reprogrammed for a different assay test.

In certain examples, the NFC chip may be programmed via arithmetic coding as a novel approach to compressing data for use with the NFC chip disclosed herein. The modified arithmetic coding algorithm described herein encodes an entire file as a sequence of symbols into a single decimal number. Typically, a string of characters (i.e., data) is represented using a fixed number of bits per character (e.g., ASCII code). When a string is converted to arithmetic encoding, frequently used characters or symbols will be stored with fewer bits and less frequently occurring characters will be stored with more bits, resulting in fewer total bits used overall. The characters or input symbols are processed at each iteration. The initial interval [0, 1) (or [0, 1]) is successively divided into subintervals on each iteration according to the probability distribution of the characters or symbols. The subinterval that corresponds to the input character or symbol is selected for next iteration. The interval derived at the end of this division process is used to decide the label or codeword for the entire sequence of characters or symbols.

Attachment 1 contains an exemplary machine-readable version of code used to generate the NFC reaction tube tag. Referencing Attachment 1, for example, the ‘assay’ may comprise a field named ‘temp_profile’ followed by a ‘max_duration’ field of type duration interval of_8(2, −9, 0.0), which is a duration, in seconds, represented by an 8 bit floating point value with 2 bits for the exponent, an exponent bias of −9, and a DC offset of zero. For the 8 bit floating value, 256 values may be represented as shown in Table 1 below (e.g., using the modified technique in the present disclosure, the values are all integers).

TABLE 1

| | | | | | | | | | | | | | | | |
|---|---|---|---|---|---|---|---|---|---|---|---|---|---|---|---|
| 0 | 16 | 32 | 48 | 64 | 80 | 96 | 112 | 128 | 144 | 160 | 176 | 192 | 208 | 224 | 240 |
| 256 | 272 | 288 | 304 | 320 | 335 | 352 | 368 | 384 | 400 | 416 | 432 | 448 | 464 | 480 | 496 |
| 512 | 528 | 544 | 560 | 576 | 592 | 608 | 624 | 640 | 656 | 672 | 688 | 704 | 720 | 735 | 752 |
| 768 | 784 | 800 | 816 | 832 | 848 | 864 | 880 | 896 | 912 | 928 | 944 | 960 | 976 | 992 | 1008 |
| 1024 | 1040 | 1056 | 1072 | 1088 | 1104 | 1120 | 1135 | 1152 | 1168 | 1184 | 1200 | 1216 | 1232 | 1248 | 1264 |
| 1280 | 1296 | 1312 | 1328 | 1344 | 1360 | 1376 | 1392 | 1408 | 1424 | 1440 | 1450 | 1472 | 1488 | 1504 | 1520 |
| 1535 | 1552 | 1568 | 1584 | 1600 | 1616 | 1632 | 1648 | 1664 | 1680 | 1696 | 1712 | 1728 | 1744 | 1760 | 1776 |
| 1792 | 1808 | 1824 | 1840 | 1856 | 1872 | 1888 | 1904 | 1920 | 1936 | 1952 | 1968 | 1984 | 2000 | 2016 | 2032 |
| 2048 | 2080 | 2112 | 2144 | 2176 | 2208 | 2240 | 2272 | 2304 | 2336 | 2368 | 2400 | 2432 | 2464 | 2496 | 2528 |
| 2560 | 2592 | 2624 | 2656 | 2688 | 2720 | 2752 | 2784 | 2816 | 2848 | 2880 | 2912 | 2944 | 2976 | 3008 | 3040 |
| 3072 | 3104 | 3135 | 3168 | 3200 | 3232 | 3264 | 3296 | 3328 | 3360 | 3392 | 3424 | 3456 | 3488 | 3520 | 3552 |
| 3584 | 3616 | 3648 | 3680 | 3712 | 3744 | 3776 | 3808 | 3840 | 3872 | 3904 | 3935 | 3968 | 4000 | 4032 | 4064 |
| 4096 | 4160 | 4224 | 4288 | 4352 | 4416 | 4480 | 4544 | 4608 | 4672 | 4735 | 4800 | 4864 | 4928 | 4992 | 5056 |
| 5120 | 5184 | 5248 | 5312 | 5376 | 5440 | 5504 | 5568 | 5632 | 5696 | 5760 | 5824 | 5888 | 5952 | 6016 | 6080 |
| 6144 | 6208 | 6272 | 6335 | 6400 | 6464 | 6528 | 6592 | 6656 | 6720 | 6784 | 6848 | 6912 | 6976 | 7040 | 7104 |
| 7168 | 7232 | 7296 | 7360 | 7424 | 7488 | 7552 | 7616 | 7680 | 7744 | 7808 | 7872 | 7935 | 8000 | 8064 | 8128 |

The NFC data strings may be concatenated with a separator and compressed. An example encoded NFC tag is shown below in Example 1. The encoded NFC tag was generated by the code described in Attachment 1, as initialized by the program fragment shown in Attachment 2. Currently available NFC chips are limited to 144 bytes of storage thus limiting the amount of data that may be stored on the chip. By using the modified compression technique described above, however, the inventors were surprisingly able to store more parameters on the NFC chip resulting in the capability to conduct far more analytical tests using the detector than previously allowed.

### Example 1

test.nfc:

| | | | | | | | | | | | | | | | | | |
|---|---|---|---|---|---|---|---|---|---|---|---|---|---|---|---|---|---|
| 00000000 | 0a | 0c | 87 | 85 | a4 | 43 | 3c | 28 | 01 | 70 | de | 0a | ba | b1 | 40 | 66 | .....C<(.p....@f |
| 00000010 | 01 | eb | df | 13 | be | b7 | c4 | f0 | 01 | f0 | e3 | 80 | 7c | 38 | eb | 47 | ............\|B.G |
| 00000020 | 91 | 6f | d9 | 3c | a4 | 6a | 75 | a3 | c8 | b7 | ec | 9e | a4 | 67 | 3a | d1 | .o.<.ju......g:. |
| 00000030 | e4 | 5b | f6 | 4f | 7b | 4d | 9d | 68 | f2 | 2d | fb | 27 | c9 | 33 | 4e | 08 | .[.O[M.h. [symbol] . [symbol] .3N. |
| 00000040 | 13 | 01 | 00 | 12 | 10 | 01 | 11 | 00 | 0c | 90 | 0e | f3 | ba | fd | 63 | 9c | ..............c. |
| 00000050 | c3 | aa | 78 | 76 | a8 | c1 | f0 | b8 | 1c | 04 | e2 | 61 | 5d | 79 | 5d | df | ..xv.......a]y]. |
| 00000060 | 3c | 8b | f3 | 96 | ef | 29 | 00 | fa | 59 | a3 | 89 | ef | e3 | f3 | 35 | 3c | <....)..Y.....5c |
| 00000070 | 14 | 21 | ca | 31 | 99 | 21 | 25 | e0 | c6 | 8b | ef | | | | | | .\|.1.\|%.... |

[symbol]

indicates data missing or illegible when filed

In an example embodiment, the NFC chip may be attached to a removable rim for reaction tube 110, and while a first reaction tube 110 may be discarded after a use, the removable rim may be removed and used for a second reaction tube 110. Thus, NFC chips may be readily reused for multiple assay runs. Using NFC chips allows for a straightforward approach for automating tagging reaction tube 110 (e.g., using NFC chips alleviates sticking/unsticking barcodes or QR codes to reaction tube 110 every time a new data is needed to tag reaction tube 110, using scanner tools for scanning barcodes or QR codes, and analyzing QR codes via image analysis algorithms). It should be understood, however, that in other embodiments the NFC chip may be permanently coupled to the reaction tube and the reaction tube with NFC chip may be disposable. In some instances, disposable NFC chips may be preferred, for example, in clinical settings, where it can be desirable to minimize the risk of setting incorrect parameters or misidentifying a sample. In other instances, reusable/reprogrammable NFC chips may be preferred, for example, in a developmental setting, where frequent and rapid parameter changes may occur.

In various embodiments, receiver 120 is configured to receive/emit a communication signal (e.g., a near-field communication signal, a variable electric or magnetic field, radio-frequency electromagnetic waves, and the like) from near-field communication chip 115 coupled to the reaction tube. In some cases, receiver 120 causes the near-field communication signal of chip 115 to emit the radio-frequency electromagnetic waves (e.g., by exciting/wirelessly powering chip 115). As used herein, near-field communication (NFC) includes any suitable approach in which a tag or chip, interrogated by an electromagnetic field, transmits a radio-frequency response that includes stored (programmed and/or configured) information identifying the tag. Thus, as used herein, NFC includes RFID tags and other similar technology. Typically, an NFC tag will be unpowered (i.e., will not require a battery or other source of energy to respond to the interrogation signal. In some instances, Bluetooth® or other (typically powered) communication technologies can be substituted for NFC-based communications described herein.

In various embodiments, near-field communication signal is configured to communicate data 116 which includes information for determining a parameter associated with a type of test to be performed on the analyte by device 105. In an example embodiment a parameter may include a wavelength of light 153 (or a plurality of wavelengths of light 153), a duration of time for illuminating sample 111, an intensity of light 153, a time delay between emission of light 153 and detection of light 155, a spatial distribution of light 153 over sample 111 (e.g., by controlling one or more optical elements associated with light emitting source 152, as described above), or any other suitable parameter related to light 153. Further, the parameter may also include a type of sensor used by optical detector 154, a duration of time for exposing optical detector 154 to light 155, a location (e.g., height along well 130) or at which light 155 is detected. Other parameters include a temperature of sample 111 (which, as further discussed below may be controlled by a suitable heating element), additives used in preparing sample 111, amount of sample 111 in reaction tube 110 (e.g., the amount of sample 111 may be measured by determining a weight of reaction tube 110), and the like.

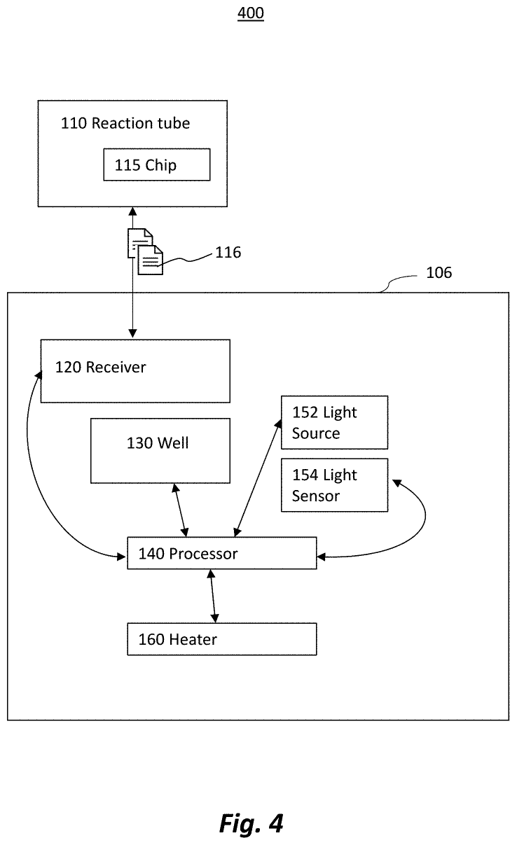

FIG. 4 shows another embodiment of a device 106 that includes a heater 160 (herein also referred to as a heat block 160). In various embodiments, device 106 is the same as device 105 but with an addition of heater 160. In an example embodiment, heater 160 is configured to control a temperature of sample 111. For example, heater 160 may be configured to surround reaction tube 110 to cause sample 111 to reach a specific temperature (e.g., 60-150 F). In various embodiments, heater 160 may be controlled by processor 140, and data 116 received by receiver 120 may or be used to determine how heater 160 is to be controlled for a particular test performed by device 106. For example, processor 140 may be operably coupled to heat block 160 and receiver 120, and processor 140 may be configured to control a temperature of heat block 160 based on various parameter such that the analyte in sample 111 is amplified. Further processor 140 is configured to determine at least one of a quantity or a concentration of the analyte in sample 111 after amplifying the analyte. FIG. 4 also shows that reaction tube 110 and device 106 form a system 400 for performing a test of sample 111.

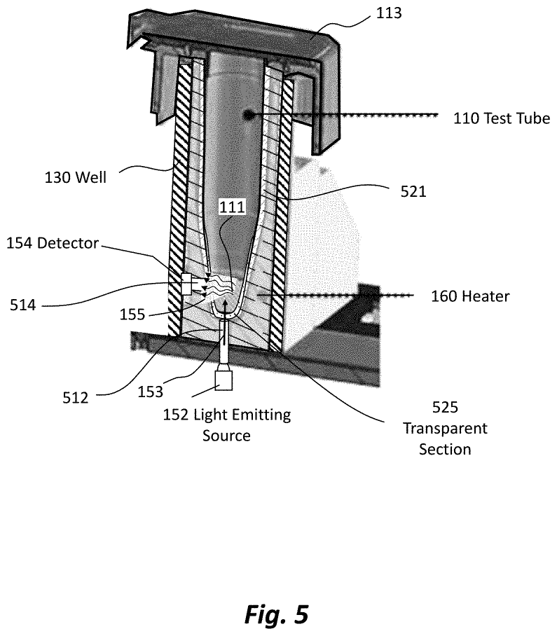

An example reaction tube 110, and well 130 is shown in FIG. 5. Reaction tube 110's walls 521 may be transparent to light 153 and light 155. Alternatively, only a portion of tube 110 may be transparent to light 153 and light 155. For example, walls 525, as shown in FIG. 5, may be transparent to light 153 and light 155 (light 153 and/or light 155 may be visible light, near infrared light, or ultraviolet light). For example, walls 525 may be made of light transparent plastic, glass, or any other suitable light transparent material. In an example embodiment walls 525 are adjacent to sample 111 located in reaction tube 110. In some cases, a bottom of reaction tube 110 may be transparent to light 153 and a side portion of reaction tube 110 may be transparent to light 155.

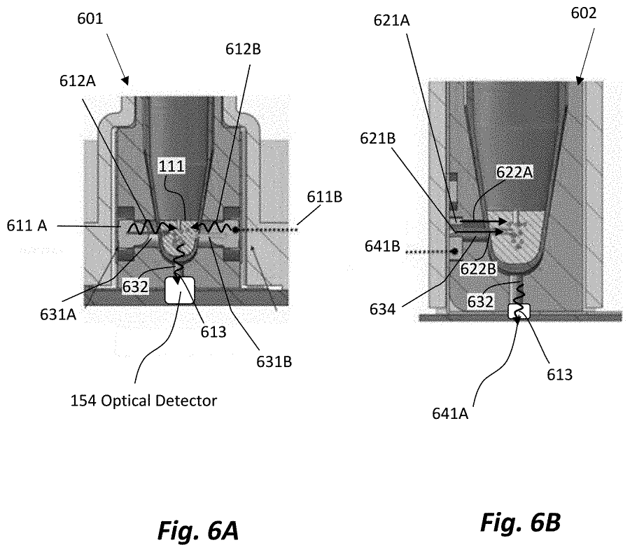

In an example embodiment, reaction tube 110 is inserted in well 130 in an opening within well 130. The walls of the opening may be surrounded by heater 160, and heater 160 may be configured to heat at least a portion of reaction tube 110 (e.g., the portion of the reaction tube containing sample 111). For example, heater 160 may be configured to heat walls 525. Heater 160 may be formed of a solid material containing heating elements. In an example embodiment, heating elements may be resistive heating elements. In various embodiments, heater 160 may include light guiding channels 512 and 514 capable of light guiding. For example, channel 512 is configured to light guide light 155 emitted by sample 111, and channel 514 is configured to light guide light 153 emitted by a light emitting source 152. In an example embodiment, guiding channels 512 and 514 may be openings. In some cases, openings 512 and 514 may include reflective walls configured to reflect respective lights 153 and 154. Alternatively, guiding channels 512 and 514 may be formed from any suitable material transparent to respective lights 153 and 155. For example, guiding channels 512 and 514 may be formed from optical fibers transparent to respective lights 153 and 155. In an example embodiment, guiding channels 512 and 514 may be made from any suitable material (e.g., glass, quarts, sapphire, light transparent plastic, and the like). As shown in FIG. 5, guiding channel 512 is adjacent to light emitting source 152, and guiding channel 514 is adjacent to optical detector 154. Placing light emitting source 152 underneath reaction tube 110 is one possible configuration and other possible locations may be used, as shown, for example, in FIGS. 6A and 6B.

FIG. 6A shows an example embodiment of a well 601 including a first light emitting source 611A configured to emit light at a first wavelength 612A (herein, for brevity we refer to this light as light 612A), and a second light emitting source 611B is configured to emit light at a second wavelength 612B (herein, for brevity we refer to this light as light 612B). First wavelength 612A may be, for example, a blue light (e.g., light 612A may have a wavelength of 400-520 nm) and second wavelength 612A may be a yellow light (e.g., light 612B may have a wavelength of 565-590 nm). It should be noted that any other suitable wavelengths for lights 612A and 612B may be used. In an example embodiment, light 612A may be light guided towards a wall of reaction tube 110 via channel 631A and light 612B may be light guided towards a wall of reaction tube 110 via channel 631B. FIG. 6A shows an embodiment, in which optical detector 154 is located underneath reaction tube 110 and light 613 emitted from sample 111 may reach optical detector 154 via channel 632. In various embodiments, as described above, channels 631A, 631B and 632 may be openings or may be formed from material transparent to light 612A, 612B and 613.

FIG. 6B shows another embodiment of well 601, containing a channel 634 for transmitting light 622A and 622B emitted by respective sources 621A and 621B. In an example embodiment, light 622A have a first peak wavelength, and light 622B may have a second wavelength different from the first wavelength. For instance, the first wavelength may correspond to a substantially green light and a second wavelength may correspond to a substantially red light. In an example embodiment, well 601 may include several optical sensors, such as sensors 641A and 641B. In an example embodiment, optical sensors 641A and 641B may be placed at different locations. For example, sensor 641A may be placed underneath reaction tube 110 and may receive light 613 via a channel 632. Additionally, optical sensor 641B may be placed adjacent to light emitting sources 621A and 621B and may receive light emitted by sample 111 via channel 634.

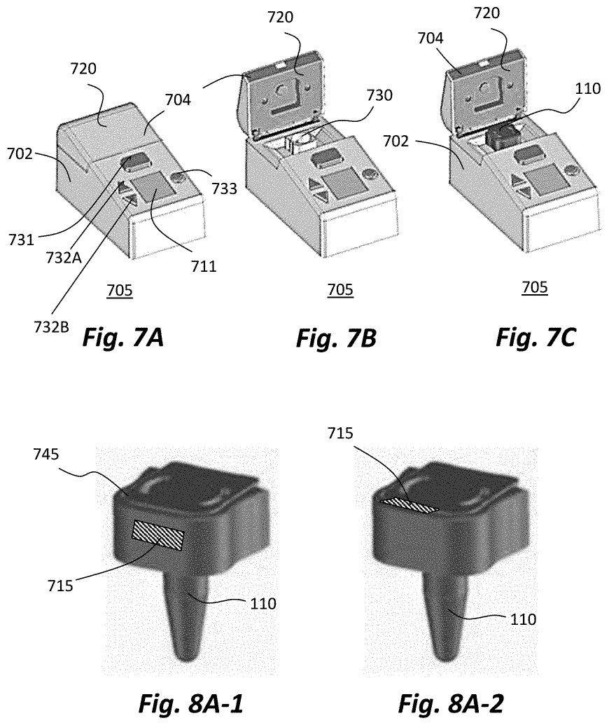

FIGS. 7A-7C show an example embodiment of device 705 (herein also referred to as a Fluorescence of Loop Primer Upon Self Dequenching Loop-Mediated Isothermal Amplification (FLOS-LAMP)) device which may be substantially the same as device 106, as shown in FIG. 4. Device 705 may include a body 702 configured to receive reaction tube 110. Cover 704 can be coupled to body 702, as shown in FIG. 7A. In an example embodiment, cover 704 may be a hinged cover (or any other suitable cover attached in any other suitable way to device 705) configured to be movable to cover a top portion of reaction tube 110. In some cases, cover 704 is configured to contain (and, in some cases, lock) the reaction tube 110 into the body 702. Further, receiver 720 (receiver 720 may be the same as receiver 120, as shown in FIG. 1, as described above), may be located within cover 704 and in proximity to chip 115 of reaction tube 110.

FIG. 7A also shows a display screen 711 configured to display information during and after performance of the assay. Further device 705 may have a cover button 731 for opening cover 704. Further, there may be a select button for selecting options displayed on screen 711, and up and down respective buttons 732A and 732B for moving up or down between options on the screen. In various embodiments, the options may be associated with a type of assay that is being performed.

Underneath cover 704 body 702 may include a well 730 for placing reaction tube 110, as shown in FIG. 7B. FIG. 7C shows reaction tube 110 placed in well 730 of section 702. In various embodiments, device 705 is configured to amplify an analyte using Polymer Chain Reaction (PCR), Loop-Mediated Isothermal Amplification (LAMP), Real-Time FLOS (RT-LAMP), Fluorescence of Loop primer upon self-dequenching (FLOS LAMP, or any other suitable techniques. Device 705 can further be configured to measure a signal associated with a quantity of the analyte, according to an embodiment. For example, device 705 may be configured to selectively amplify polynucleotide sequence(s). For example, the reaction tube 110 can include suitable primers to selectively cause one or more analytes and/or one or more controls in the sample to be amplified according to known techniques (e.g., LAMP, etc.).

In various embodiments, reaction tube 110 is configured to be inserted in an enclosure of a device (e.g., enclosure 133, as shown in FIG. 2A) for performing a test to determine at least one of a quantity or a concentration of an analyte in sample 111. In various embodiments, reaction tube 110 includes a body portion closed at a bottom portion, the bottom portion being at least partially transparent to excitation light at an excitation wavelength (e.g., light 153, as shown in FIG. 3A) and to emission light at an emitted wavelength (e.g., light 155, as shown in FIG. 3B). Further, reaction tube 110 includes a tube top member opposite the bottom portion (e.g., rim 113, as shown in FIG. 5). In an example embodiment, the tube top member includes communication chip 115 configured to communicate a communication signal to receiver 120. In an example embodiment, the communication signal includes parameters configured to instruct device 105 (as shown in FIG. 1) how to conduct an assay of the analyte. In some the top member of the tube may include a cover for reaction tube 110, the cover configured to contain (or lock) the analyte in the reaction tube 110

In some cases, the top member is configured to attach to a top portion of reaction tube 110. For example, the top member may be one of a screw top, a crown cap, a snap on top, or a friction fit cap.

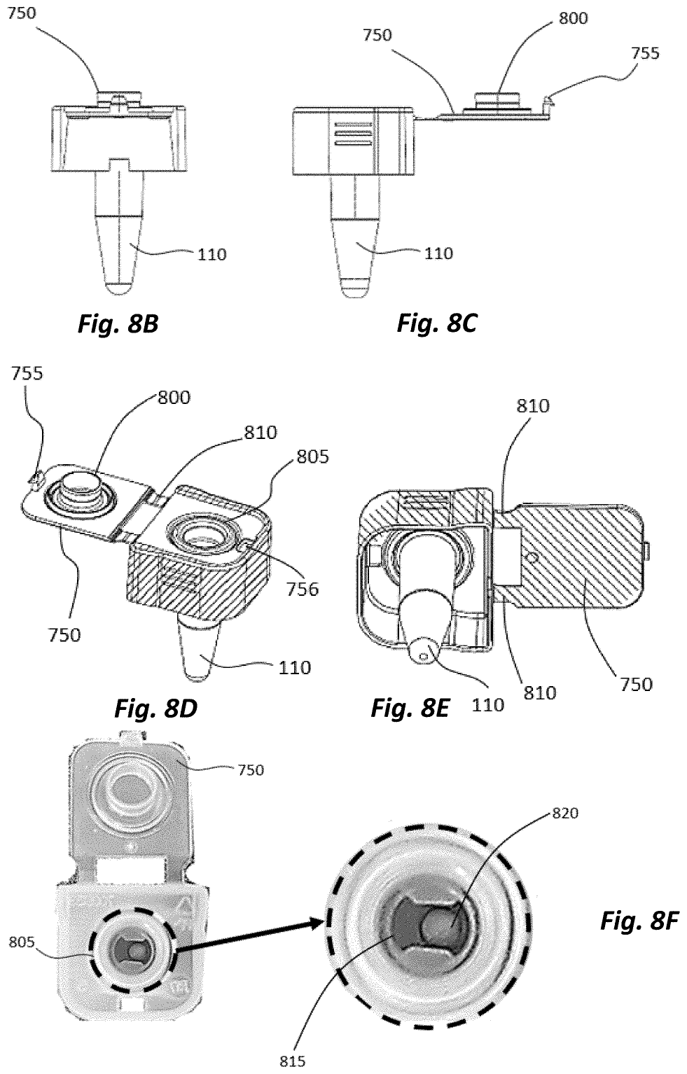

FIGS. 8A-1 and 8A-2 show reaction tubes 110 including a communication chip 715. In an example embodiment, as shown in FIG. 8A-1, chip 715 may be located on a side of reaction tube 110, and in another embodiment, as shown in FIG. 8A-2, chip 715 may be located on a top portion 745 of reaction tube 110. It should be appreciated that chip 715 may be placed at any portion of external surfaces of reaction tube 110 (e.g., chip 715 may be or include a sticker that may be attached to any suitable portion of external surfaces of reaction tube 110).

FIGS. 8B-8F show another example embodiment of reaction tube 110 disclosed herein. Reaction tube 110 may include a lid or cover 750. Lid or cover 750 may include lock 755 that may be configured to releasably engage lock slot 756 to lock or secure an analyte or sample in reaction tube 110. Alternatively, lock 755 may me configured to permanently secure lid or cover 750 to lock slot 756. Cover or lid 750 may include an extension 800 configured to engage upper receptacle 805. Extension 800 and upper receptacle 805 may include at least one or multiple grooves and/or ridges to form a friction fit to secure lid or cover 750 thus keeping an analyte or sample in reaction tube 110. In some examples, the reaction tube 110 may not include lock 755. In some examples, cover or lid 750 may include hinge 810. In other examples, cover or lid 750 may include at least two hinges 810.

FIG. 8F shows upper receptacle 805 and a magnified view of upper receptacle 805. In the magnified figure, keeper slot 815 is positioned in the center of receptacle 805. Keeper slot 815 may be configured to prevent a solid object within reaction tube 110 from falling out. In some examples, keeper slot 815 may be generally barbell-shaped. In other examples, the keeper slot 815 is configured to allow a liquid sample or analyte to be deposited into the reaction tube 110. In another example, keeper slot 815 secures a prepositioned lyophilized pellet 820 within the reaction tube 110. Lyophilized pellet 820 may include at least one biological or chemical reagent configured to react with an analyte or sample. For example, lyophilized pellet 820 may include a biological or chemical reagent that is easily reconstituted into solution form to react with the analyte or sample upon addition of a fluid to dissolve the lyophilized pellet 820. In some examples, lyophilized pellet 820 may include a plurality of biological or chemical reagents.

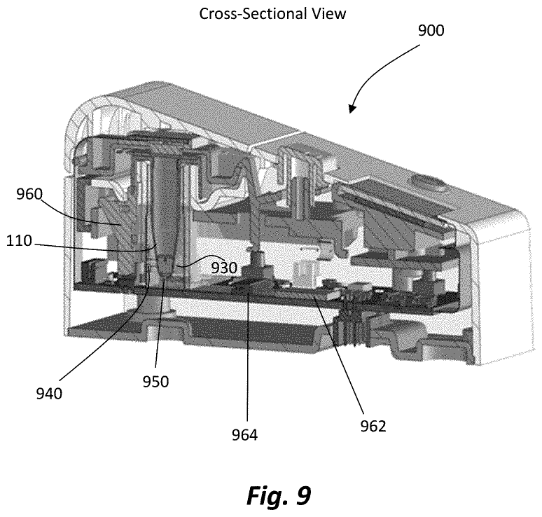

FIG. 9 is a cross sectional view of device 900 in the closed configuration with the reaction tube 110 disposed within. A heating block 960 is configured to control the temperature of the reaction tube and sample 111, for example, to maintain the temperature of analyte within sample 111 (containing appropriate primers) to cause the analyte to undergo isothermal amplification. Heating block 960 may be part of well 930 and may include a suitable channel that provides an optical pathway such that a light source 940 (e.g., light emitting diodes, lasers, etc.) can illuminate the sample and/or excite fluorescent dyes contained within the reaction tube 110. Another channel through the heating block 930 is configured to provide an optical pathway such that a sensor 950 (e.g., photodiodes, photomultipliers, charge coupled devices (CCDs) and/or any other suitable optical detectors) can detect optical signals, such as fluorescent signals emitted by fluorescent dyes and associated with a quantity and/or concentration of analyte(s) and/or control(s). In other embodiments, device 90 contains any other suitable sensor operable to detect signals characteristic of quantity and/or concentration of analytes and/or controls, such as optical sensors configured to detect native fluorescence, absorbance, and/or color (change), electrochemical sensors, pH sensors, or any other sensor operable to detect a signal indicative of a concentration and/or quantity of an analyte.

In various embodiments, device 900 includes a processor 962 and/or a memory 964. Processor 962 can be, for example, a general-purpose processor, a Field Programmable Gate Array (FPGA), an Application Specific Integrated Circuit (ASIC), a Digital Signal Processor (DSP), and/or the like. Processor 962 can be configured to retrieve data from and/or write data to memory, e.g., memory 964, which can be, for example, random access memory (RAM), memory buffers, hard drives, databases, erasable programmable read only memory (EPROMs), electrically erasable programmable read only memory (EEPROMs), read only memory (ROM), flash memory, hard disks, floppy disks, cloud storage, and/or so forth.

In various embodiments, Processor 962 and memory 964 can be communicatively coupled to heating block 930, light source 940 and/or sensor 950 and configured to control a run during which analytes and/or controls disposed within the reaction tube 110 are selectively amplified and/or analyzed. Processor 962 and memory 964 can be operable to receive, process, and/or record signals associated with concentrations of analytes and/or controls. Further, processor 962 and memory 964 are configured to determine whether the sample contained within the reaction tube 110 is “positive” or “negative” for one or more analytes, according to methods described in further detail herein. Although shown within a housing of instrument 110, in other embodiments, processor 162 and/or memory 164 can be disposed in another device. Similarly stated, instrument 110 can be communicatively coupled to an external compute device configured to control a run and/or determine whether the sample is positive or negative.

In some cases, device 900 may be configured to analyze samples in a plurality of reaction tubes, using well 930. The plurality of reaction tubes can be inserted serially, or in parallel (e.g., according to an embodiment, device 900 can include multiple wells (not shown)). In an example embodiment, each reaction tube from the plurality of reaction tubes may be configured to be inserted into well 930 of device 900. In some cases, an associated mechanism may be used to automatically insert a reaction tube into well 930, perform an analysis of a sample located in the reaction tube based on data communicated from a communication chip associated with the reaction tube, and remove the tube from well 930, thus, vacating well 930, so that device 900 may use well 930 to process a next reaction tube. In other cases, reaction tubes can be manually inserted and removed from well 930.

In an example embodiment, a first reaction tube from the plurality of reaction tubes may include a first communication chip configured to transmit a first signal including a first plurality of parameters for performing a first assay. Further, a second reaction tube from the plurality of reaction tubes may include a second communication chip configured to transmit a second signal including a second plurality of parameters for performing the second assay, at least one of the second plurality of parameters being different from at least one of the first plurality of parameters such that the second assay is different from the first assay. For example, the first plurality of parameters may include a first temperature for the heating block 960 and a first wavelength for light emitting source 940, while the second plurality of parameters may include a second temperature for the heating block 960 and a second wavelength for light emitting source 940. Other parameters may be different between the first and the second plurality of parameters (e.g., an amount of time that a sample in a reaction tube is being illuminated by a light from light emitting source 940).

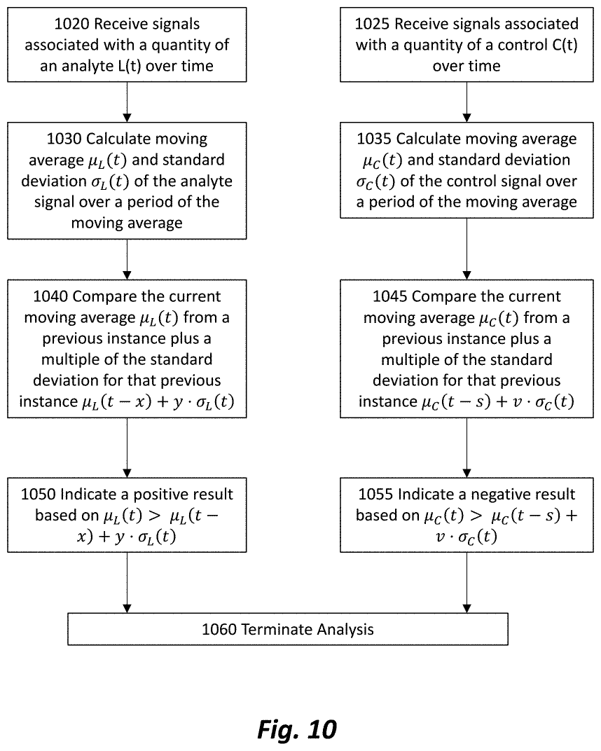

FIG. 10 is a flow chart of a method of detecting an analyte, according to an embodiment. The method shown and described in FIG. 10 can be performed by device 900 shown and described with reference to FIG. 9 or any other suitable instrument configured to selectively amplify an analyte. Throughout the method, device 900 can analyze a sample over time. For example, device 900 can be configured to perform LAMP to amplify an analyte, such as a characteristic sequence of the SARS-CoV-2 genome. During the analysis, device 900 can continuously receive signal(s) associated with a quantity of the analyte, at step 1020. Device 900 can be configured to receive a signal associated with a quantity of the analyte every second, every 5 seconds, every 10 seconds, every 20 seconds, or any other suitable sampling rate. For example, in FLOS-LAMP techniques, a labeled loop probe can be configured to fluoresce when bound to the analyte such that the intensity (L) of light emitted from the fluorophore label can be used to determine a quantity and/or concentration of the analyte as the sample is selectively amplified. The signals received at step 1020 can represent time-series data for the intensity of the fluorophore and/or concentration of the analyte.

Device 900 (or a compute device coupled to device 900) can be operable to process the signal(s) associated with the analyte that are received at step 1020. Device 900 can calculate a moving average of the intensity (μ_{L}) and a standard deviation of the intensity (σ_{L}), at step 1030. Typically, the moving average and the standard deviation will have the same window. The width of the moving average and moving standard deviation windows can be predetermined and/or dynamic. For FLOS-LAMP signals, a suitable fixed window over which the moving average and/or standard deviation are calculated can be at least or about 3 minutes, at least or about 2 minutes, at least or about 60 seconds, at least or about 30 seconds, at least or about 20 seconds, or any suitable length of time. In some embodiments, the window over which the moving average and/or standard deviation are calculated can be a function of elapsed time and/or temperature of the amplification reaction such that, for example, the length of the window decreases as the run progresses.

In other embodiments, device 900 can further process the intensity measurement to calculate a quantity of the analyte. The moving average and moving standard deviation can then be calculated for the quantity of the analyte, rather than for the intensity of the fluorophore.

Calculating the moving average and the moving standard deviation produces a data set that can be stored in memory such that a data set includes, for each instance of time t, an instantaneous intensity L(t), an average intensity over a period of time ending at the instance μ_{L}(t), and a standard deviation of intensity measurements taken over the period of time ending at that instance σ_{L}(t). Moving average and moving standard deviations can be calculated substantially in real time (e.g., within less than a second) as the intensity measurements are made. Once calculated, at step 1040, a moving average of the intensity can be compared to a sum of the moving average of the intensity calculated for a previous instance in time and a multiple of the standard deviation of the intensity calculated for that previous instance in time

μ_{L}(t−x)+y*σL(t−x) (equation 1)

where x represents the difference in time between the current instance and the previous instance; and y is a constant or function by which the moving standard deviation is multiplied.

For FLOS-LAMP analyses, a suitable x is about 8 minutes, about 6 minutes, about 4 minutes, about 2 minutes, or any other suitable time. A suitable y is 1.2, 1.5, 1.8, 2, 2.5, 3, 4, or any other suitable value. Similarly stated, for a FLOS-LAMP analysis a current (e.g., most recently calculated) moving average of intensity can be compared to the moving average of the intensity calculated 4 minutes previously plus 2 times the standard deviation of intensity calculated 4 minutes previously. At step 1040, the current moving average of intensity may be analyzed to determine whether it is greater than the sum of the moving average of the intensity calculated for a previous instance in time and a multiple of the standard deviation of the intensity calculated for that previous instance in time

μ_{L}(t)>μ_{L}(t−x)+y*σ_{L}(t−x) (equation 2).

If μ_{L}(t)>μ_{L}(t−x)+y*σ_{L}(t−x), then such a condition is defined as a target indicator. The target indicator can represent a positive result, or a presence of the analyte in the sample. In some instances, upon determining that a sample is positive, a signal indicating the positive result can be immediately (e.g., within 3 seconds) sent (e.g., to a user or technician), at step 1050, and/or the sample run can be terminated at 1060. In other instances, the indication of a positive result can be sent based on the target indicator for a period of time (e.g., 5 seconds, 10 second, 15 seconds, 30 seconds, etc.). In this way, samples can be continuously be evaluated for positivity during amplification and the run can be terminated upon detecting a positive result, which can eliminate the need to amplify the sample for a predetermined period of time and evaluating the sample after processing. Such a technique can, in many instances, result in much shorter run times compared to known methods.

In some instances, a similar technique can be applied to a control signal to detect negative results (e.g., the absence of the analyte from the sample and/or the sample containing a quantity of the analyte that is below a detection threshold). The sample can contain one or more internal controls and fluorescent tags configured to produce a luminescent signal indicative of a quantity of the controls. Typically, the sample will contain a control with a known initial quantity and/or concentration. Device 900 can be configured to selectively amplify the control(s) simultaneously with the analytes such that, given a known initial quantity/concentration of a control, the time for that control to indicate can be predicted. As discussed in further detail herein the sequence and/or difference in time between the control indicating and the analyte indicating can be used to determine whether the sample is positive or negative. Therefore, the initial quantity/concentration of the control can be associated with a detection threshold of the analyte. The sample can also include additional controls configured to indicate if the sample run fails for various reasons. For example, a control can be used to determine whether a sufficient volume of sample was obtained. RNaseP, which is known to be present in predictable concentrations in human nasal mucous, can be used to evaluate whether a sufficient volume of human nasal mucous sample is present. The failure of an RNaseP control to indicate (e.g., before a control having a known concentration indicates) can therefore cause device 900 to send a signal indicating that the test was inconclusive for insufficient sample.

In some embodiments, the internal control may be endogenous to the sample, e.g., a biological sample, or the internal control may be added to the sample. In one non-limiting example, when detecting the presence of viral DNA or RNA in a biological sample, the internal control may be RNA expressed from a housekeeping gene or ribosomal RNA. Typically, the luminescent signal(s) indicative of the quantity of the control(s) will have a different spectral and/or temporal characteristic than the luminescent signal indicative of the quantity of the analyte. In other instances, the sample can be subdivided into two or more subsamples. Each subsample can be configured to be analyzed for one or more different analytes and/or serve as a control for one or more different analytes. In such an embodiment, each subsample would typically be amplified simultaneously. During the analysis of the sample, device 900 can continuously receive signal(s) associated with a quantity of the control, at step 1025.

Device 900 (or the compute device coupled to device 900) can be operable to process the signal(s) associated with the control that are received at step 1025. Device 900 can calculate a moving average of the intensity of the control signal μC and a standard deviation of the intensity of the control signal σ_{C}, at step 1035. In other embodiments, device 900 can further process the intensity measurement to calculate a quantity of the control. The moving average and moving standard deviation can then be calculated for the quantity of the control, rather than for an intensity associated with the quantity of the control.

Calculating the moving average and the moving standard deviation produces a data set that can be stored in memory such that a data set includes, for each instance of time t, an instantaneous intensity of the control signal C(t), an average intensity of the control signal over a period of time ending at the instance μ_{C}(t), and a standard deviation of intensity of the control signal taken over the period of time ending at that instance σ_{C}(t). At step 1045, the current moving average of intensity of the control signal may be analyzed to determine whether it is greater than the sum of the moving average of the intensity of the control signal calculated for a previous instance in time and a multiple of the standard deviation of the intensity calculated for that previous instance in time

μ_{C}(t)>μ_{L}(t−s)+v*σ_{C}(t−s) (equation 3).

Here s represents the difference in time between the current instance and the previous instance; and v is a constant or function by which the moving standard deviation is multiplied). If μ_{C}(t)>μ_{C}(t−s)+v*σ_{C}(t−s), then such a condition is defined as a control indicator. For FLOS-LAMP analyses, a suitable s is about 8 minutes, about 6 minutes, about 4 minutes, about 2 minutes, or any other suitable time. A suitable v is 1.2, 1.5, 1.8, 2, 2.5, 3, 4 or any other suitable value. In some instances, s can equal x (from equations 1 and/or 2) and/or v can equal y (from equations 1 and/or 2). In other instances, constants/functions used to determine whether the control indicates can be different from constants/functions used to determine whether the target indicates. For example, x can equal 240 seconds, y can equal 2, s can equal 360 seconds, and v can equal 1.5. In addition, or alternatively, the windows over which the moving averages and standard deviations for the target and control can be the same or different.

In some instances, if the control indicates and the target does not indicate, a signal indicating a negative result can be sent, at step 1055 and/or the sample run can be terminated, at step 1060. In some embodiments, the negative result can be sent at step 1055 and/or the sample run can be terminated at step 1060 immediately (e.g., within 3 seconds) of the control indicating in the absence of the sample indicating. In other embodiments, upon the control indicating, the run can continue for a fixed period of time (e.g., 3 minutes, 5 minutes, or any other suitable time period) or for a dynamically determined period of time that is a function of, for example, time since the initiation of the run. If the target indicates during the period of time after the control indicates, a signal indicating a positive test result can be sent at step 1055 and/or the run can be terminated at step 1060. In yet other embodiments, upon the target indicating, the run can continue for a fixed period of time (e.g., 3 minutes, 5 minutes, or any other suitable time period) or for a dynamically determined period of time that is a function of, for example, time since the initiation of the run. If the control indicates during the period of time after the target indicates, a signal indicating a negative test result can be sent at step 1055 and/or the run can be terminated at step 1060. In instances in which more than one control is used, the absolute and/or relative timing at which each control and/or target indicates can be used to determine whether to send an indication of a positive result or a negative result.

In some embodiments, an indication of a positive test result and/or negative test result is ignored (e.g., not analyzed for, suppressed, not sent, reported, logged, and/or the basis for terminating a run) if it occurs in an initial portion of the analysis. In a LAMP analysis, a sample is typically inserted into a pre-heated heater-block. Typically, fluorophore characteristics cause the target and control signal to be weaker at lower temperatures (e.g., before the sample reaches thermal equilibrium with the heater-block). Such weaker signals may not be reliable indications of sample positivity/negativity. In addition, the rate at which fluorophore intensity increases as the sample nears thermal equilibrium typically decreases. Similarly stated during an initial portion of the sample run when the sample is approaching thermal equilibrium with the heater block, the target and control signals are typically rising and concave-down. Thus, in some instances, positive test results and/or negative test results can be ignored if the target and/or control signals, respectively, have a positive slope and negative concavity. Slope and concavity measurements of the target and/or control signals can be based on a time-windowing queue, similar to the moving averages and moving standard deviations discussed above. Once a negative slope or positive concavity for the target and/or control signal is detected, indications for that signal may no longer be ignored. In other instances, positive test results and/or negative test results can be ignored for a predetermined fixed time period. For example, target indications can be ignored during the first 180, 240, 300, 360, 420 seconds, or any other suitable time period, of the run. As another example, control indications can be ignored during the first 630, 690, 750, 810, 870 seconds, or any other suitable time period.

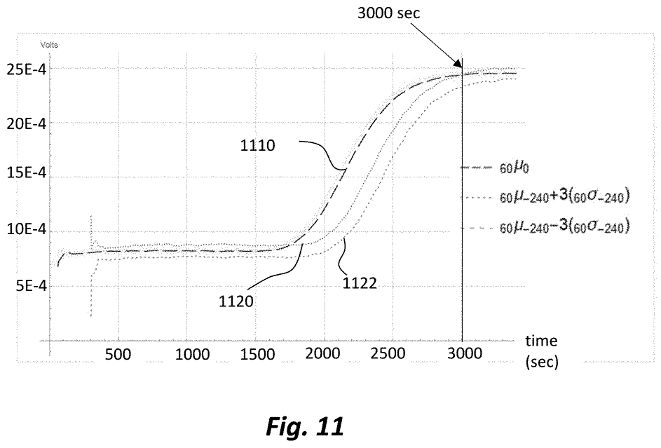

FIG. 11 is an experimental data from a FLOS-LAMP analysis of an example sample. Line 1110 represents a moving average of the intensity of fluorophore that is associated with a quantity of a target analyte. Lines 1120 and 1122 represent the moving average of the intensity of the fluorophore offset in time+/−a multiple of a standard deviation of the intensity of the fluorophore, respectively. In this instance, the offset is 240 seconds and the multiple of the standard deviation is 3. Therefore, line 1120 represents μ_{L}(t−240)+3σ_{L}(t−240), and line 1122 represents μ_{L}(t−240)−3σ_{L}(t−240). At approximately 3000 seconds from run initiation, line 1110 and line 1120 cross, such that μ_{L}(3000)>μ_{L}(3000−240)+3σ_{L}(3000−240), representing the target indicating. Thus, at approximately 3000 seconds, a signal indicating a positive result can be sent and, optionally, the run can be terminated. Alternatively, the run can proceed for an additional period of time to assure that the target continues to indicate.

In various embodiments, a device for analyzing an analyte (e.g., device 105, as shown in FIG. 1) is configured to perform more than one assays (e.g., device 105 may be configured to perform assays other than only COVID-19 assays). For each assay, there may be multiple parameters that characterize the correct execution of the assay by device 105. In an example embodiment, the determined parameters are stored on a chip (e.g., chip 115) associated with a reaction tube (e.g., reaction tube 110). In some cases, instead of chip 115 another tag may be used, as described above (e.g., QR code or other suitable barcode or the like). The chip and/or tag can be read by a receiver of the device, and parameters can be sent to a processor and/or memory of the device such that when the device performs an assay, the assay is performed according to the specified parameters.

One parameter associated with an assay that can be stored by the tag and read and applied by a device is temperature. Temperature can include a set temperature, maximum temperature, minimum temperature, and/or a temperature profile (e.g., how temperature should vary over time during the assay). For example, the temperature profile may be characterized by a duration for holding a first setpoint temperature following commencement of the assay. A second setpoint temperature may be specified for a reminder portion of the analysis of the analyte, and a third setpoint temperature may be specified following a completion of the analysis.

Another example parameter may include a maximum duration of time needed for analyzing the analyte before calling an analysis as either negative or inconclusive (e.g., maximum duration may be from a few minutes to a few hours, depending on a type of analysis). For example, some techniques for analyzing an analyte, such as the analysis shown and described with reference to FIG. 10, may tend to produce false or misleading results in the initial portion of an analysis. Accordingly, signals produced during an initial portion of a run may not be analyzed for positive/negative results and/or positive/negative results may be suppressed during an initial portion of the run. A parameter specifying the maximum duration of time needed for analyzing the analyte may indicate the duration of the initial portion of the run during which signals are not analyzed.

Additional parameters may include a loop cycle time for reading all analytes when multiple analytes are analyzed. The loop cycle time may be, for example, one to a few seconds. Also, a delay between reading analytes within one loop cycle may be used as a parameter. The delay may be a millisecond, for example. A total number of analytes (control or target) contained in assay may be used as a parameter (for a target analyte there may be a control analyte).

Additional parameters may be associated with an excitation light that should be used for analyzing the analyte (e.g., parameters may include a mean wavelength of light, a standard deviation, full width at half maximum (FWHM), and the like). Additionally, or alternatively, a parameter may identify a particular light emitting source for exciting of an analyte (e.g., in embodiments in which multiple light emitting sources, such as light emitting devices (LEDs) with different emission spectra.) Further, a parameter may identify a power setting for a light emitting source (e.g., a resultant luminosity and/or an applied voltage or current). In addition, or alternatively, an illumination profile can be parameterized. For example, in a manner similar to temperature profile parameterization discussed above, instructions as to how the illumination of the reaction tube should very over time (including, for example, intensity and/or wavelength) can be stored in on the chip and/or tag. In some cases, a parameter may specify that more than one power source may be used simultaneously or sequentially. For example, a first LED emitting light at a first wavelength that is configured to excite a sensing moiety configured to emit a signal that is dependent on a concentration of an analyte, while a second LED emitting light at a second wavelength is configured to excite a control moiety that emits a signal that is independent of the analyte (e.g., a constant signal or a signal that increases at a known or expected rate). It should be appreciated that any number of excitation of light sources available to device 105 may be identified by a suitable parameter and/or their outputs may be parameterized (e.g., over time)

Additional parameters may be associated with emission wavelength(s) from the analyte. In some cases, an expected emission frequency for the analyte (including mean values and standard deviation) may be specified as parameters, and based on that information, a proper detecting device (or several detecting devices) may be selected by device 105.

Other non-exclusive list of parameters that may be stored on chip 115 (or which can be read from a QR code associated with reaction tube 110) may include, an algorithm used for analyzing the analyte. For example, an algorithm may use different excitation sources, detectors, temperatures, etc. for analyzing the analyte, and collect data at various time windows. For instance, the light emission data from the analyte may be collected after an excitation LED is turned off (or between the pulses of light provided by the excitation LED). Further parameters may include indication for initiating procedures for determining system malfunction (e.g., procedures for determining system malfunction may initiate a set of tests for determining sensitivity of detectors, operational powers of excitation sources, as well as proper temperature control for the assay). Various tests may be performed including placing a control assay (i.e., the assay with controlled properties) and analyzing the assay to ensure proper operation of device 105.

In various embodiments, during a performance of the assay (or after the completion of the analysis of an analyte) device 105 may display on a screen (e.g., screen 711, as shown in FIG. 7A) associated with a device results for the analysis. For example, the results may include the identification of the pathogen that was (or was not) detected. For example, screen 711 may display “COVID-19 Virus Detected,” or “Flu Virus Detected,” or “RSV Virus Detected,” or “COVID-19 Virus Not Detected,” or other similar information. Other parameters displayed by screen 711 may include those which are technical in nature. For example, such parameters may include a duration of analysis after which an “inconclusive” is called, or the reaction tube temperature at which the reaction executes. In various embodiments, parameters characterizing the result of the assay will vary based on the parameters for the assay that are stored on chip 115 (or similar device, as described above)

In some cases, a device 105 may not be suitable to perform an assay specified by a chip 115. For example, when device 105 is not capable of performing an assay as specified by parameters stored by chip 115 (e.g., cannot reach specified temperature, cannot emit specified excitation signal, cannot detect specified emission signal, etc.), the reaction tube 110 tagged with such parameters may be rejected by device 105. Device 105 may display an error message if an incorrect type of reaction tube is inserted. In some cases, only reaction tubes that have appropriate tags (the tags may be stored on chip 115 or elsewhere on reaction tube) may be accepted for the analysis. Further, in some implementations, reaction tubes with improperly configured chip 115, or absence of chip 115 may not be analyzed.

While chip 115 is convenient for storing parameters associate with the analysis of an analyte, additionally (or alternatively) reaction tube 110 may also have at least some parameters that are readily available to be read by a human. For instance, in addition to the presence of chip 115, reaction tube 110 may have a suitable tag. Having both machine and human read parameters allows for a human verification of parameters during the analysis procedure, thus reducing a possibility of an error associated with a test, especially in point-of-care (POC) workflows. In an example embodiment, chip 105 may be tagged via chip 115 to do a COVID-19 analysis, and an associated readable tag on a reaction tube may read “COVID-19.” Then during the analysis, device 105 may display on the screen that the COVID-19 analysis is being performed, and a human operator may confirm that the right analysis is being performed.

In an example embodiment, for a particular assay, there may be an associated identifier (human readable and/or encoded). The identifier may not be unique over space and time; that is, it may not be a serial number. It is anticipated that hundreds of millions of assays may be performed, each with its own reaction tube. If the identifier needed to be globally unique, it would thus require ten or more decimal digits. Such large identifiers are unwieldy. Instead, the operational requirement of reduction sample mix-up in a workflow can be achieved by ensuring that (with very high probability) no two reaction tubes processed at the same time by a given facility share the same identifier. A workable approach may be to use six-digit identifiers that are assigned in a defined sequence, which would ensure that identifier reuse occurs only after a million reaction tubes have been produced.

In addition to the pre-applied numbering on the reaction tube, each test may also contain a small selection of matching-numbered stickers that be peeled off and applied to other paper documentation involved in the workflow.

In some cases, device 105 may facilitate performing assays that are simultaneously analyzes a sample for multiple analytes. For example, an assay might check for any of COVID, Flu, and RSV. In some cases, device 105 may support “one-of-n” style multiplexed tests. It may not necessarily need to support “all-of-n” tests. For example, if a patient is infected with both COVID and Flu, a COVID-plus-Flu test need only indicate that the patient has COVID or that the patient has Flu; it need not indicate that the patient has both COVID and Flu. Alternatively, device 105 may be configured to determine that assay is positive for both COVID and Flu.

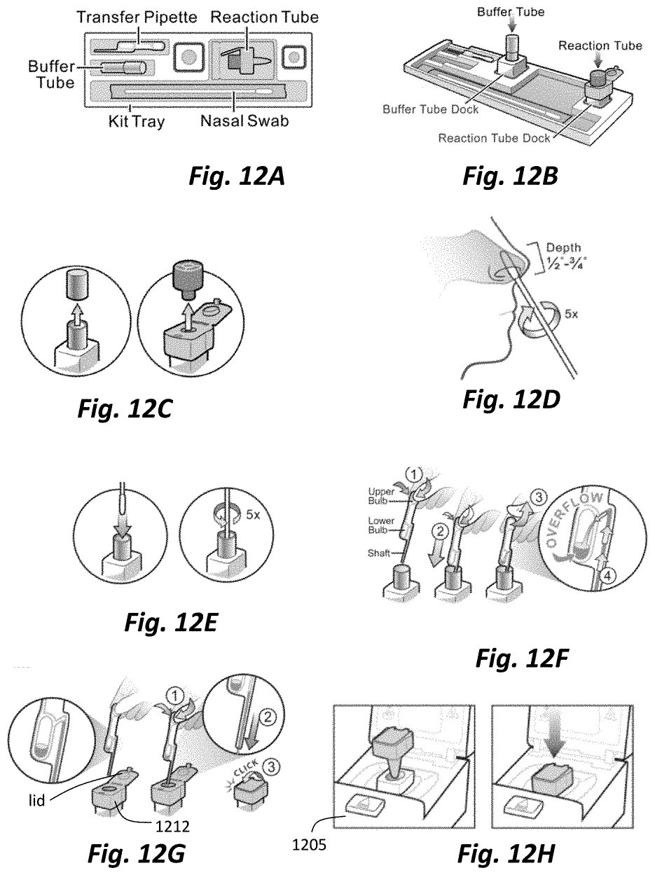

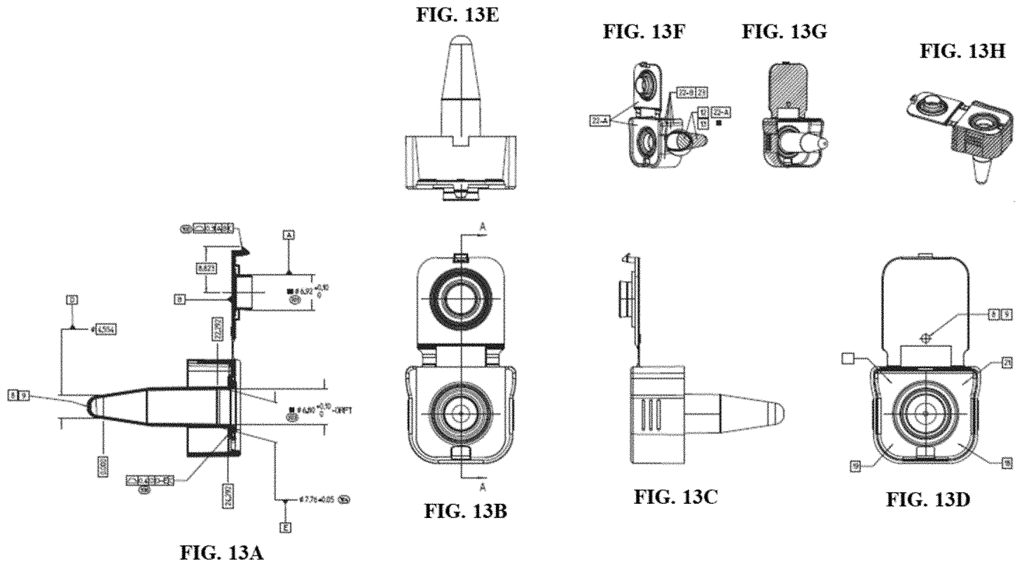

In an example embodiment, device 105 and reaction tube 110, as shown in FIG. 1, may be part of a test kit. An example test kit is shown in FIGS. 12A-12H. In FIG. 12A a kit may have components that include a nasal swab, a buffer tube, a transfer pipette, and a reaction tube, and a set of instructions that may include the steps described below. The components may be placed in a kit tray, as shown in FIG. 12A. In an example embodiment, buffer tube and a reaction tube may be positioned within the kit tray, as shown in FIG. 12B, and may be opened at about the same time, as shown in FIG. 12C. In an example embodiment, nasal swab may be used to swab a patient's nostrils and then be inserted into the buffer tube, as shown in FIG. 12D. The nasal swab may swipe the first nostril by executing 5 full rotations at a depth of about ½ to ¾ of an inch. Then a second nostril may be swabbed with the same nasal swab in the same way.

The buffer tube is configured to contain a suitable solution for preparing the assay. After material from the nasal swab is released into the solution contained in the buffer tube (as shown in FIG. 12E), the pipette may be used to extract the solution sample from the buffer tube, as shown in FIG. 12F. The pipette may have upper and lower bulbs. A user may first squeeze the upper bulb to release air from the pipette and then release the upper bulb to obtain the solution sample. The lower bulb may be used to store an excess of the solution, as shown in FIG. 12F. A user then may release the sample into the reaction tube by squeezing the upper bulb, as shown in FIG. 12G (note that a fixed amount of the sample solution is configured to be release from the pipette into the reaction tube due to a particular design of the pipette having a lower bulb). FIG. 12G shows that the reaction tube includes a top portion 1212 (e.g., top portion 1212 may be the same as top portion 745, as shown in FIG. 8A) which may be configured to have a lid for closing top portion 745, as shown in FIG. 12G. Subsequently, the reaction tube may be placed into a device for analyzing an analyte as shown in FIG. 12H, and as describing above.

In various embodiments, quality control procedures may be performed to test that a device 1205 (as shown in FIG. 12H) is performing correctly. A negative and a positive control test may be performed. The negative control test includes performing all the steps of the test of the sample solution without having the nasal swab swabbing nostrils of a patient. If device 1205 operates correctly, the negative control test should result in negative detection of pathogens (e.g., a negative detection of COVID-19 virus).

A positive control test includes a sample containing the pathogen (e.g., the Microbiologics Helix Elite™ Synthetic Standard, SARS-CoV-2 Process Control (Pellet), Catalog No. HE0062S). In an example embodiment, performing the positive control test includes performing all the steps of the test of the sample solution with the nasal swab inserted into the solution containing a pathogen. In various cases, to avoid contamination, a negative control test may be run prior to running a positive control test.

In an example embodiment, it may be preferred that both negative and control tests are run at least once for each first-time operator, at the beginning of each day of testing, before using a new lot or shipment of test kits, when problems with the test kits or a detector of device 1205 are suspected, or as deemed necessary to conform internal quality control procedures, local state and/or federal regulations, or regulations for accrediting groups. Testing patient samples should only be performed after both controls have been run and determined to produce the expected results. If the expected control results are not obtained, the failed positive and/or negative control test may be repeated using a new test kit. If the failure repeats, patient sample testing should not be repeated, and device should be further tested and repaired by a qualifying party. Further, the samples tested by the device used since the last successful control test should be discarded.

In various embodiments, encoding test parameters on the tube reduces the level of expertise required to run the sample and reduces the likelihood that a sample will be subjected to the wrong assay (e.g., the wrong assay may result in false positive or false negative results). For example, when testing multiple assays, encoding test parameters on chips of various test tubes first and performing tests later may reduce the number of errors that otherwise might happen when processing one test tube at a time. Further, encoding test parameters on an array of tubes allows for processing a large number of tests automatically.

While various embodiments have been described above, it should be understood that they have been presented by way of example only, and not limitation. Where schematics and/or embodiments described above indicate certain components arranged in certain orientations or positions, the arrangement of components may be modified. While the embodiments have been particularly shown and described, it will be understood that various changes in form and details may be made. Although various embodiments have been described as having particular features and/or combinations of components, other embodiments are possible having a combination of any features and/or components from any of the embodiments as discussed above.

For example, although methods described herein generally relate to FLOS-LAMP analyses and are particularly well suited to SARS-CoV-2 detection, it should be understood that the techniques described herein can be applied to many other analyte and/or target detection schemes. Similarly stated, embodiments described herein are not limited to SARS-CoV-2 detection but can be applied to any analyte that can be selectively amplified or concentrated, through, for example, LAMP, polymerase chain reaction (PCR), chemical synthesis, electrochemistry, bioproduction, chromatography, electrophoresis, isoelectric focusing, gravimetric separation, etc. Although embodiments described herein generally describe fluorescent signals that are associated with or can be correlated to a quantity or concentration of an analyte, analytes can be detected by any suitable means such as, for example, a pH-driven colorimetric signal from Real-Time Loop-Mediated Isothermal Amplification (RT-LAMP), or any other suitable colorimetric, electric, electro-chemical, optical absorbance, etc. signal. Similarly stated, the method shown and described with reference to FIG. 2 is well suited to any suitable analysis where a “positive” result is characterized by exponential or other rapid growth of a signal from a relatively low baseline.

In particular embodiments, the methods disclosed herein may be used to determine the presence or absence of an analyte by detecting and/or measuring a signal generated via PCR. A variety of different PCR methods may be used, including but not limited to: basic PCR, reverse transcriptase (RT)-PCR, Hot-start PCR, competitive PCR, or quantitative real-time (qRT)-PCR, e.g., as described at https://www.dot.promega.dot.com/resources/guides/nucleic-acid-analysis/per-amplification/ and references discussed therein.

In particular embodiments, the methods disclosed herein may be used to determine the presence or absence of an analyte by detecting and/or measuring a signal generated via isothermal nucleic acid amplification. Isothermal amplification of nucleic acids is an alternative to polymerase chain reaction (PCR). The advantage of these methods is that the nucleic acids amplification can be carried out at constant temperature, unlike PCR, which requires cyclic temperature changes. In certain embodiments, the isothermal nucleic acid amplification is performed using, e.g., loop mediated isothermal amplification (LAMP), nucleic acid sequence based amplification) (NASBA), Helicase dependent amplification (HDA), Exponential amplification reaction of nucleic acids (EXPAR), Strand displacement amplification (SDA), Recombinase polymerase amplification (RPA), rolling circle amplification (RCA), e.g., as described in O. L. Bodulev1 and I. Yu. Sakharov, Biochemistry (Moscow), 2020, Vol. 85, No. 2, pp. 147166 and references cited therein, which is incorporated by reference herein in its entirety.

Parameters shown and described above with reference to FIG. 10 (e.g., the window for the moving average, the window for the standard deviation, the temporal offset x, and the standard deviation multiple y) are generally described in the context of FLOS-LAMP and are selected based on the characteristic shape of positive target and/or control signals. A skilled data scientist, taking the above into account could readily select other appropriate parameters for signals having different characteristics.

In particular embodiments, the analyte is an infectious agent or a pathogen, or a component thereof. In particular embodiments, the infectious agent or pathogen is a virus, bacteria, or a fungus. In particular embodiments, the infectious agent is an influenza virus or a coronavirus, e.g., SARS-CoV-2. In some embodiments, methods disclosed herein are used to determine the presence of the infectious agent or pathogen by detecting presence of infectious agent DNA or RNA, e.g., in a sample. In some embodiments, the sample is a biological sample obtained from a subject diagnosed with the infection or considered to be at risk of having or developing the infection. In other embodiments, the sample is a food product or beverage product. In some embodiments, the sample is obtained from a surface, e.g., a food preparation surface, a food or beverage package surface, or a surface in a home, rental home, or hotel, such as but not limited to a kitchen counter surface, a bathroom counter surface, a toilet, shower, or bathtub surface, or a table or dresser surface.

In certain embodiments, the analyte is a virus or component thereof. In some embodiments, the sample is a biological sample obtained from a subject diagnosed with or suspected of being or at risk of being infected with the virus. In particular embodiments, the virus is a norovirus, rotavirus, adenovirus, astrovirus, influenza virus, coronavirus, parainfluenza virus, respiratory syncytial virus, human immunodeficiency virus (HIV), human T lymphotropic virus (HTLV), rhinovirus, hepatitis A virus, hepatitis B virus, Epstein Barr virus, or West Nile virus. In particular embodiments, the virus is SARS-CoV-2.

In certain embodiments, the virus is an influenza virus, including but not limited to any of the types or subtypes, lineages, or clades disclosed herein. There are four types of influenza viruses: A, B, C and D. Human influenza A and B viruses cause seasonal epidemics of disease (known as the flu season) almost every winter in the United States. Influenza A viruses are the only influenza viruses known to cause flu pandemics, i.e., global epidemics of flu disease. Influenza type C infections generally cause mild illness and are not thought to cause human flu epidemics. Influenza D viruses primarily affect cattle and are not known to infect or cause illness in people.

Influenza A viruses are divided into subtypes based on two proteins on the surface of the virus: hemagglutinin (H) and neuraminidase (N). There are 18 different hemagglutinin subtypes and 11 different neuraminidase subtypes (H1 through H18 and N1 through N11, respectively). Current subtypes of influenza A viruses that routinely circulate in people include: A(H1N1) and A(H3N2). Certain circulating influenza A(H1N1) viruses are related to the pandemic 2009 H1N1 virus that emerged in the spring of 2009 and caused a flu pandemic. This virus, scientifically called the “A(H1N1)pdm09 virus,” and more generally called “2009 H1N1,” has continued to circulate seasonally since then. Influenza A(H3N2) viruses have formed many separate, genetically different clades in recent years that continue to co-circulate.

Influenza B viruses are not divided into subtypes, but instead are further classified into two lineages: B/Yamagata and B/Victoria.

In certain embodiments, the virus is a coronavirus, including but not limited to any of the types or subtypes or groupings disclosed herein. Coronaviruses are named for the crown-like spikes on their surface. There are four main sub-groupings of coronaviruses, known as alpha, beta, gamma, and delta. Seven coronaviruses that can infect people are: the common human coronaviruses: 229E (alpha coronavirus); NL63 (alpha coronavirus); OC43 (beta coronavirus); HKU1 (beta coronavirus); and other human coronaviruses: MERS-CoV (the beta coronavirus that causes Middle East Respiratory Syndrome, or MERS); SARS-CoV (the beta coronavirus that causes severe acute respiratory syndrome, or SARS); and SARS-CoV-2 (the novel coronavirus that causes coronavirus disease 2019, or COVID-19. Humans commonly are infected with human coronaviruses 229E, NL63, OC43, and HKU1.

In certain embodiments, the virus is SARS-CoV-2. A new disease called coronavirus disease 2019 (COVID-19) has been reported. COVID-19 is caused by infection with the novel coronavirus, SARS-CoV-2 or 2019-nCoV. In some embodiments, the analyte is detected in a biological sample obtained from a subject diagnosed with or is considered at risk of having or developing COVID-19.

In certain embodiments, the analyte is a bacterium or component thereof. In some embodiments, the sample is a biological sample obtained from a subject diagnosed with or considered at risk of having a bacterial infection. In certain embodiments, the bacterium is one of the following: Acinetobacter, Bacteroides, Burkholderia, Clostridium, Enterobacteriaceae, Enterococcus, Klebsiella, Staphylococcus, Streptococcus, Morganella, Mycobacterium, Neisseria, Pseudomonas, or Stenotrophomonas, including any of the following: Acinetobacter baumannii, Bacteroides fragilis, Burkholderia cepacia, Clostridium difficile, Clostridium sordellii, Carbapenem-resistant Enterobacteriaceae), Enterococcus faecalis, Klebsiella pneumonia, Staphylococcus aureus, including Methicillin-resistant Staphylococcus aureus (MRSA) and Vancomycin-resistant Staphylococcus aureus), Morganella morganii, Mycobacterium abscessus, Pseudomonas aeruginosa, Stenotrophomonas maltophilia, Mycobacterium tuberculosis, Streptococcus pneumonia, Neisseria meningitidis, or Vancomycin-resistant Enterococci.

In certain embodiments, the analyte is a fungus. In particular embodiments, the sample is a biological sample obtained from a subject diagnosed with or is considered at risk of having or developing a fungal infection. In certain embodiments, the fungus is any of the following: Aspergillus, Candida (including Candida auris), Cryptococcus neoformans, Pneumocystis (including Pneumocystis jirovecii), Mucormycetes, Taloromyces, Candida, Blastomyces, Coccidioides, Histoplasma, Cryptococcus (including Cryptococcus gattii), or Paracoccidioides.

In addition, some methods disclosed herein describe terminating a sample run when a target or control indicates (optionally, after a waiting period). It should be understood, however, that in other embodiments, the sample can be run (e.g., the target analyte can be selectively amplified) to a maximum duration (e.g., 60 minutes, 90 minutes, etc.). In such an embodiment, an indication of a positive result can be sent if the target has indicated in that time period (optionally accepting indications during an excluded initial period). An indication of a negative result can be sent if the control has indicated in the time period. In other scenarios, a signal indicating that the test has failed or is indeterminate can be sent.

Where methods and/or events described above indicate certain events and/or procedures occurring in a certain order, the ordering of certain events and/or procedures may be modified. Additionally, certain events and/or procedures may be performed concurrently in a parallel process when possible, as well as performed sequentially as described above.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. The drawings are not necessarily to scale, and those skilled in the art will recognize that variations in relative dimensions can vary. For example, for ease of illustration, FIG. 2A-3D generally exaggerate the space between the walls defining the sample well and the reaction tube. In some implementations it can be desirable for a reaction tube and a sample well to form a sliding fit with the walls of the reaction tube in contact with, or spaced less than 1 mm apart from the walls of the sample well, as this can facilitate even heat transfer, in embodiments in which the reaction tube is configured to be heated. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto; inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The above-described embodiments can be implemented in any of numerous ways. For example, embodiments of the present technology may be implemented using a combination of hardware, and software (or firmware). When implemented in firmware and/or software, the firmware and/or software code can be executed on any suitable processor or collection of logic components, whether provided in a single device or distributed among multiple devices.

In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the invention discussed above. The computer readable medium or media can be transportable, such that the program or programs stored thereon can be loaded onto one or more different computers or other processors to implement various aspects of the present invention as discussed above.

The terms “program” or “software” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present invention need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of” “only one of” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The terms “substantially,” “approximately,” and “about” used throughout this Specification and the claims generally mean plus or minus 10% of the value stated, e.g., about 100 would include 90 to 110.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

ATTACHMENT 2

```
public string NfcTest1(string fileName) {
  KaitaiStream ks = new KaitaiStream(fileName, FileAccess.ReadWrite);
  AnavasiNfc rootWrite = new AnavasiNfc(ks);
  // rootWrite.NfcSerialNumber.SetRandom( );
  rootWrite.NfcSerialNumber.DisplayString = “120F-0TWZ-UHSM”;
  AnavasiNfc.Nfc nfc = rootWrite.Top;
  nfc.Header.Version.Number.IntValue = 0;
  var stepWarm = new AnavasiNfc.TempStep(ks);
```

| | |
|---|---|
| stepWarm.Duration.Seconds.FloatValue = 3 * 60; | // ANALYSIS_TUBE_HEATER_HOT_TRANSITION_SECS |
| stepWarm.TubeSetpoint.Degrees.FloatValue = 57.0; | // TUBE_TEMPERATURE_WARM_SETPOINT_CENTICELSIUS |
| stepWarm.TubeTolerance.Degrees.FloatValue = 1.5; | // TUBE_TEMPERATURE_TOLERANCE_CENTICELSIUS |
| stepWarm.LidSetpoint.Degrees.FloatValue = 70; | // LID_TEMPERATURE_WARM_SETPOINT_CENTICELSIUS |
| stepWarm.LidTolerance.Degrees.FloatValue = 2.5; | // LID_TEMPERATURE_TOLERANCE_CENTICELSIUS |

var stepHot = new AnavasiNfc.TempStep(ks);

| | |
|---|---|
| stepHot.Duration.Seconds.FloatValue = 0; | // indefinite duration |
| stepHot.TubeSetpoint.Degrees.FloatValue = 63.0; | // TUBE_TEMPERATURE_HOT_SETPOINT_CENTICELSIUS |
| stepHot.TubeTolerance.Degrees.FloatValue = 1.5; | // TUBE_TEMPERATURE_TOLERANCE_CENTICELSIUS |
| stepHot.LidSetpoint.Degrees.FloatValue = 100.0; | // LID_TEMPERATURE_HOT_SETPOINT_CENTICELSIUS |
| stepHot.LidTolerance.Degrees.FloatValue = 2.5; | // LID_TEMPERATURE_TOLERANCE_CENTICELSIUS |

```
  nfc.Assay.TempProfile.AddStep(stepWarm);
  nfc.Assay.TempProfile.AddStep(stepHot);
  nfc.Assay.TempProfile.AnalysisSettling.Seconds.FloatValue = 2 * 60; // ANALYSIS_SETTLING_SECS -
  ANALYSIS_TUBE_HEATER_HOT_TRANSITION_SECS
  nfc.Assay.TempProfile.TubeMalfunctionTemp.Degrees.FloatValue = 100.0;
  nfc.Assay.TempProfile.LidMalfunctionTemp.Degrees.FloatValue = 110.0;
  nfc.Assay.TestFlavor = AnavasiNfc.MvpTestFlavor.Covid19;
  nfc.Assay.MaxDuration.Seconds.FloatValue = 3900.0;
  nfc.Assay.ImpossiblyShortNegativeDuration.Seconds.FloatValue = 18 * 60;
  nfc.Assay.LoopCycleTime.Seconds.FloatValue = 1.0;
  nfc.Assay.DelayBetweenAnalytes.Seconds.FloatValue = 0.1;
  nfc.Assay.Leds[0].Current.FloatValue = 3000;
  nfc.Assay.Leds[0].Gain = AnavasiNfc.As7341Gain.Gain64;
  nfc.Assay.Leds[0].Atime.IntValue = 39;
  nfc.Assay.Leds[0].Astep.FloatValue = 999;
  nfc.Assay.Leds[1].Current.FloatValue = 11000;
  nfc.Assay.Leds[1].Gain = AnavasiNfc.As7341Gain.Gain128;
  nfc.Assay.Leds[1].Atime.IntValue = 39;
  nfc.Assay.Leds[1].Astep.FloatValue = 999;
  var sunrise = new AnavasiNfc.Sunrise(ks);
  sunrise.InitialSamplesToIgnore.Seconds.FloatValue = 180;
  sunrise.SampleAveragingWindow.Seconds.FloatValue = 60;
  sunrise.LaggingTimeOffset.Seconds.FloatValue = 360;
  sunrise.LaggingConstraintSdFactor.FloatValue = 1.9;
  sunrise.LaggingConstraintMin.FloatValue = 0.0003;
  sunrise.MinExcursionDuration.Seconds.FloatValue = 15;
  nfc.Assay.FluorophoreSignalType = AnavasiNfc.FluorophoreSignalType.FourChan;
  nfc.Assay.Fluorophores[0].Sunrise_ = sunrise;
  nfc.Assay.Fluorophores[1].Sunrise_ = sunrise;
  nfc.Assay.Fluorophores[2].Sunrise_ = sunrise;
  nfc.Assay.Fluorophores[3].Sunrise_ = sunrise;
  nfc.Assay.Fluorophores[0].IgnoreSaturationDuration.Seconds.FloatValue = 180;
  nfc.Assay.Fluorophores[1].IgnoreSaturationDuration.Seconds.FloatValue = 180;
  nfc.Assay.Fluorophores[2].IgnoreSaturationDuration.Seconds.FloatValue = 180;
  nfc.Assay.Fluorophores[3].IgnoreSaturationDuration.Seconds.FloatValue = 180;
  nfc.Assay.Fluorophores[0].MinSignal.FloatValue = 0.005;
  nfc.Assay.Fluorophores[1].MinSignal.FloatValue = 0.010;
  nfc.Assay.Fluorophores[2].MinSignal.FloatValue = 0.015;
  nfc.Assay.Fluorophores[3].MinSignal.FloatValue = 0.020;
  nfc.Assay.Fluorophores[0].MaxSignal.FloatValue = 0.05;
  nfc.Assay.Fluorophores[1].MaxSignal.FloatValue = 0.10;
  nfc.Assay.Fluorophores[2].MaxSignal.FloatValue = 0.15;
  nfc.Assay.Fluorophores[3].MaxSignal.FloatValue = 0.20;
  var analyte0 = new AnavasiNfc.Analyte(ks);
  var analyte1 = new AnavasiNfc.Analyte(ks);
  var analyte2 = new AnavasiNfc.Analyte(ks);
  var analyte3 = new AnavasiNfc.Analyte(ks);
  analyte0.AnalyteType = AnavasiNfc.AnalyteType.Control;
  analyte0.GraceWindow.Seconds.FloatValue = 10 * 60;
  analyte1.AnalyteType = AnavasiNfc.AnalyteType.Target;
  analyte2.AnalyteType = AnavasiNfc.AnalyteType.Target;
  analyte3.AnalyteType = AnavasiNfc.AnalyteType.Target;
  var name1 = new AnavasiNfc.AnalyteName(ks);
  var name2 = new AnavasiNfc.AnalyteName(ks);
  var name3 = new AnavasiNfc.AnalyteName(ks);
  // First string is *always* the test name
  nfc.Assay.Strings.AddString(“Covid & Flu”);
  var istr0 = nfc.Assay.Strings.AddString(“Influenza”);
  var istr1 = nfc.Assay.Strings.AddString(“Gonorrhea”);
  var istr2 = nfc.Assay.Strings.AddString(“Hepatitis”);
  name1.AnalyteNameType = (AnavasiNfc.AnalyteNameType)istr0;
  name2.AnalyteNameType = (AnavasiNfc.AnalyteNameType)istr1;
  name3.AnalyteNameType = (AnavasiNfc.AnalyteNameType)istr2;
  analyte1.AnalyteName = name1;
  analyte2.AnalyteName = name2;
  analyte3.AnalyteName = name3;
  var and0 = new AnavasiNfc.FluorophorePatternAnd(ks);
  var and1 = new AnavasiNfc.FluorophorePatternAnd(ks);
  var and2 = new AnavasiNfc.FluorophorePatternAnd(ks);
  var and3 = new AnavasiNfc.FluorophorePatternAnd(ks);
  and0.Match.IntValue = 1;
  and1.Match.IntValue = 2;
  and2.Match.IntValue = 4;
  and3.Match.IntValue = 8;
  analyte0.FluorophorePatterns.Add(and0);
  analyte1.FluorophorePatterns.Add(and1);
  analyte2.FluorophorePatterns.Add(and2);
  analyte3.FluorophorePatterns.Add(and3);
  nfc.Assay.AddAnalyte(analyte0);
  nfc.Assay.AddAnalyte(analyte1);
  nfc.Assay.AddAnalyte(analyte2);
  nfc.Assay.AddAnalyte(analyte3);
```

1. A device comprising:
a well configured to receive a reaction tube containing an analyte;
a receiver configured to receive wirelessly transmitted data from a chip coupled to the reaction tube, the wirelessly transmitted data including information for determining a parameter associated with a type of test to be performed on the analyte by the device;
a light emitting source configured to emit an excitation light at a wavelength to illuminate the analyte in the reaction tube;
an optical detector configured to receive an emission light in response to the analyte being illuminated by the excitation light; and
a processor operably coupled to the receiver and the light emitting source, the processor configured to select the wavelength of the emission light based on the parameter.

2. The device of claim 1, further comprising:
a heat block defining the well,
the processor operably coupled to the heat block and configured to control the temperature of the analyte.

3. The device of claim 2, wherein the heat block includes a first opening configured to create an optical path from the light emitting source to a transparent portion of the reaction tube.

4. The device of claim 1, wherein the light emitting source includes one or more light emitting diodes.

5. The device of claim 1, wherein the light emitting source is configured to illuminate a bottom part of the reaction tube.

6. The device of claim 1, further comprising:
a hinged cover configured to be movable to cover a top portion of the reaction tube,
the receiver located within the hinged cover.

7. The device of claim 6, wherein the receiver is further configured to emit or receive a near-field communication signal.

8. A kit for use with the device of claim 1 comprising, a set of instructions, a nasal swab, a buffer tube, a transfer pipette, and the reaction tube.

9. A device, comprising:
a heat block defining a well configured to receive a reaction tube containing an analyte;
a receiver configured to receive a near-field communication signal from a near-field communication chip coupled to the reaction tube, the near-field communication signal including information for determining a parameter associated with a type of test to be performed on the analyte by the device;
a processor operably coupled to the heat block and the receiver, the processor configured to:
control a temperature of the heat block based on the parameter such that the analyte is amplified; and
determine at least one of a quantity or a concentration of the analyte after amplifying the analyte.

10. The device of claim 9, further comprising:
a light emitting source configured to emit an excitation light to illuminate the analyte in the reaction tube; and
an optical detector configured to receive an emission light in response to the analyte being illuminated by the excitation light,
wherein the processor is operably coupled to the receiver and the light emitting source, and configured to determine the at least one of the quantity or the concentration of the analyte by causing the analyte to be illuminated by the light emitting source and the emission light to be received and processed.

11. The device of claim 10, wherein:
the heat block includes a first opening configured to create an optical path from the light emitting source to a first portion of the reaction tube; and
the heating block includes a second opening configured to create an optical path to the optical detector from a second portion of the reaction tube.

12. A reaction tube configured to be inserted in an enclosure of a device for performing a test to determine at least one of a quantity or a concentration of an analyte, the reaction tube comprising:
a tubing closed at a bottom portion, the bottom portion being at least partially transparent to excitation light at an excitation wavelength and to emission light at an emitted wavelength; and
a tube top member opposite the bottom portion, the tube top member including a near-field communication chip configured to communicate a near-field communication signal to a receiver, the receiver being part of the device, the signal including parameters configured to instruct the device how to conduct an assay of the analyte.

13. The reaction tube of claim 12, wherein the top member includes a cover for the reaction tube, the cover configured to lock the analyte in the reaction tube.

14. The reaction tube of claim 12, wherein the bottom portion is constructed of at least one of a plastic or glass that is transparent to at least one of visible light, near infrared light, or ultraviolet light.

15. The reaction tube of claim 12, wherein the near-field communication chip is a passive radio-frequency identification tag activated by radio-frequency electromagnetic waves emitted by the receiver of the device.

16. The reaction tube of claim 12, wherein the reaction tube further includes a lyophilized pellet, and wherein the lyophilized pellet comprises at least one biological reagent.

17. The reaction tube of claim 12, wherein the top member is one of a screw top, a crown cap, a snap on top, or a friction fit cap.

18. A system including the reaction tube of claim 12, the system further comprising the device, the device including:
a well configured to receive the reaction tube;
a light emitting source configured to emit an excitation light at a wavelength to illuminate the analyte in the reaction tube; and
an optical detector configured to receive an emission light in response to the analyte being illuminated by the excitation light; and
a processor operably coupled to the receiver and the light emitting source, the processor configured to select the wavelength of the emission light based on the parameters.

19. A kit comprising the reaction tube of claim 12, a set of instructions, a nasal swab, a buffer tube, and a transfer pipette.

20. A system, comprising:
a device including:
a heat block defining a well, the heat block configured control a temperature of at least one reaction tube from a plurality of reaction tubes, wherein the at least one reaction tube further includes a cover member configured to cover a top portion of the at least one reaction tube;
a receiver positioned within the cover member and configured to receive any one of a plurality of near-field communication signals from the at least one reaction tube, each near-field communication signal from the plurality of near-field communication signals including a plurality of parameters for performing an assay on a sample contained in the at least one reaction tube;
a light emitting source configured to emit an excitation light to illuminate the sample in the reaction tube;
an optical detector configured to receive an emission signal in response to the sample being illuminated by the excitation light; and
a processor operably coupled to the receiver and configured to control the heating block, the light emitting source, and the optical detector based on plurality of parameters; and
the plurality of reaction tubes, each reaction tube from the plurality of reaction tubes configured to be inserted into the well,
a first reaction tube from the plurality of reaction tubes including a first near-field communication chip configured to transmit a first signal including a first plurality of parameters for performing a first assay,
a second reaction tube from the plurality of reaction tubes including a second near-field communication chip configured to transmit a second signal including a second plurality of parameters for performing the second assay, at least one of the second plurality of parameters being different from at least one of the first plurality of parameters such that the second assay is different from the first assay.
